(19) United States
(12) Patent Application Publication
(10) Pub. No.: US 2024/0000565 A1
(43) Pub. Date: Jan. 4, 2024

(54) PROSTHETIC HEART VALVES WITH RATCHETING LOCK MECHANISMS AND METHODS FOR FABRICATION AND USE

(71) Applicant: EDWARDS LIFESCIENCES CORPORATION, Irvine, CA (US)

(72) Inventors: Nikolai Gurovich, Hadera (IL); Michael Bukin, Pardes Hanna (IL); Anatoly Dvorsky, Haifa (IL); Eran Grosu, Kfar-Saba (IL)

(21) Appl. No.: 18/369,734

(22) Filed: Sep. 18, 2023

Related U.S. Application Data
(63) Continuation of application No. PCT/US2022/025693, filed on Apr. 21, 2022.
(60) Provisional application No. 63/178,475, filed on Apr. 22, 2021.

(51) Int. Cl. A61F 2/24

(52) U.S. Cl. CPC A61F 2/2418; A61F 2220/0025; A61F 2240/001; A61F 2250/0058

(57) ABSTRACT

A prosthetic heart valve has a frame and a valvular structure supported by the frame. The frame has cells distributed circumferentially around the frame and formed by struts. The struts includes a support arm, which has a fixed end portion extending from an axial end portion of the frame and a free end portion disposed toward an opposite axial end portion of the frame relative to the fixed end portion. The fixed end portion has a rotational position that is rotationally offset relate to that of the free end portion. When the frame is in a radially-compressed configuration, a second locking member of the struts is spaced apart from a first locking member of the free end portion of the support arm. When the frame is in the radially-expanded configuration, the second locking member engages the first locking member, thereby restricting the frame from moving from the radially-expanded configuration.

### CROSS-REFERENCE TO RELATED APPLICATIONS

This application is a continuation of International Application No. PCT/US2022/025693, filed Apr. 21, 2022, which claims priority to U.S. Provisional Application No. 63/178,475, filed Apr. 22, 2021, both of which are incorporated by reference herein.

### FIELD

The present disclosure relates to prosthetic heart valves, and in particular, to prosthetic heart valves with ratcheting lock mechanisms, as well as methods for fabricating and using such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Prosthetic valves that rely on a mechanical actuator for expansion can be referred to as “mechanically expandable” prosthetic heart valves. The actuator typically takes the form of pull cables, sutures, wires and/or shafts that are configured to transmit expansion forces from a handle of the delivery apparatus to the prosthetic valve. Mechanically expandable prosthetic heart valves can provide one or more advantages over self-expandable and balloon-expandable prosthetic heart valves. For example, mechanically expandable prosthetic heart valves can be expanded to various fully functional working diameters. Some mechanically expandable prosthetic heart valves can also be compressed after an initial expansion (e.g., for repositioning and/or retrieval). Despite the recent advancements in percutaneous valve technology, there remains a need for improved transcatheter heart valves and delivery devices for such valves.

### SUMMARY

Described herein are prosthetic heart valves comprising ratcheting locking mechanisms and methods for implanting and fabricating such prosthetic heart valves. In some implementations, one or more cells of a frame of the prosthetic heart valve include a first locking member of a ratcheting locking mechanism. When the frame is initially fabricated, the first locking member can be oriented coplanar with a plane of the corresponding cell. The first locking member is subsequently twisted so as to have an orientation crossing a plane of the cell, for example, perpendicular to the plane of the cell. In some implementations, the frame is formed of a shape memory material, and the twisting comprises shape-setting the first locking member.

In some implementations, the first locking member is a female ratchet member having teeth on separate jaws designed to engage corresponding openings or recesses of a male ratchet member of the ratcheting locking mechanism. Before the twisting, the teeth of the female ratchet member face toward each other along a direction coplanar with the cell and/or along a direction parallel to the circumferential direction of the frame. After the twisting, the teeth of the female ratchet member face toward each other along a direction perpendicular to a plane of the cell and/or along a direction parallel to the radial direction of the frame.

In a representative example, a prosthetic heart valve comprises a valvular structure and a frame. The valvular structure comprises a plurality of leaflets. The frame is configured to support the valvular structure and to move between a radially-compressed configuration and a radially-expanded configuration. The frame comprises a central longitudinal axis, a first axial end portion, a second axial end portion, a plurality of cells, and a plurality of struts. The central longitudinal axis extends from the first axial end portion to the second axial end portion. The plurality of cells are distributed circumferentially around the frame and are formed by the plurality of struts. The plurality of struts includes a support arm, a first locking member, and a second locking member. The support arm includes a fixed end portion extending from the first axial end portion and a free end portion disposed toward the second axial end portion relative to the fixed end portion and comprises the first locking member. The fixed end portion has a first rotational position and the free end portion has a second rotational position that is rotationally offset relative to the first rotational position. When the frame is in the radially-compressed configuration, the second locking member is disposed farther toward the second axial end portion and spaced apart from the first locking member. When the frame is in the radially-expanded configuration, the second locking member engages the first locking member, thereby restricting the frame from moving from the radially-expanded configuration to the radially-compressed configuration.

In another representative example, a prosthetic heart valve comprises a frame and a valvular structure. The valvular structure is supported within the frame and comprises a plurality of leaflets. The frame has a central axis and comprises a plurality of first cells arranged along a circumferential direction of the frame. Each first cell is formed by a plurality of interconnected first struts and has an inner cell disposed within an area bounded by the first struts of the first cell. Each inner cell is formed by a plurality of interconnected second struts. Each inner cell also has a first locking member at an end of a support arm and a second locking member. The support arm extends axially from a first axial end of the respective inner cell toward a second axial end of the respective inner cell that is opposite the first axial end. The second locking member is at or adjacent to the second axial end of the respective inner cell. Each first locking member has a twisted orientation such that a first part of the first locking member is disposed radially inward of the first axial end and a second part of the first locking member is disposed radially outward of the first axial end. The frame is radially compressible and expandable between a radially-compressed configuration, where the first locking member is spaced from the second locking member along an axial direction of the frame, and a radially-expanded configuration, where the first and second locking members engage with each other to lock the frame in the radially-expanded configuration.

In another representative example, a prosthetic heart valve comprises a frame and a valvular structure. The valvular structure is supported within the frame and comprises a plurality of leaflets. The frame has a central axis and comprises a plurality of first cells arranged along a circumferential direction of the frame. Each first cell is formed by a plurality of interconnected first struts. Each first cell also has a first locking member at an end of a support arm and a second locking member. The support arm extends axially from a first axial end of the respective first cell toward a second axial end of the respective first cell that is opposite the first axial end. The second locking member is at or adjacent to the second axial end of the respective first cell. Each first locking member has a twisted orientation such that a first part of the first locking member is disposed radially inward of the first axial end and a second part of the first locking member is disposed radially outward of the first axial end. The frame is radially compressible and expandable between a radially-compressed configuration, where the first locking member is spaced from the second locking member along an axial direction of the frame, and a radially-expanded configuration, where the first and second locking members engage with each other to lock the frame in the radially-expanded configuration.

In another representative example, a prosthetic heart valve comprises a frame and a valvular structure. The valvular structure is supported within the frame and comprises a plurality of leaflets. The frame has a central axis and comprises a plurality of first cells arranged along a circumferential direction of the frame. Each first cell is formed by a plurality of interconnected first struts. Each first cell also has a first locking member at an end of a first support arm and a second locking member at an end of a second support. The first support arm extends axially from a first axial end of the respective first cell toward a second axial end of the respective first cell that is opposite the first axial end. The second support arm extends axially from the second axial end of the respective first cell toward the first axial end of the respective first cell. Each of the first and second locking members has a twisted orientation. The frame is radially compressible and expandable between a radially-compressed configuration, where the first locking member is spaced from the second locking member along an axial direction of the frame, and a radially-expanded configuration, where the first and second locking members engage with each other to lock the frame in the radially-expanded configuration.

In another representative example, a prosthetic heart valve comprises a frame and a valvular structure. The valvular structure is supported within the frame and comprises a plurality of leaflets. The frame has a central axis and comprises a plurality of first cells arranged along a circumferential direction of the frame. Each first cell is formed by a plurality of interconnected first struts. The frame is radially compressible and expandable between a radially-compressed configuration and a radially-expanded configuration. The frame comprises ratchet means for locking the frame in the radially-expanded configuration.

In another representative example, a method is provided for implanting in a patient's body the prosthetic heart valve according to any of the above-described representative examples. The method comprises inserting a distal end of a delivery apparatus into vasculature of a patient. The delivery apparatus comprises an elongated shaft. The prosthetic heart valve is releasably supported within the delivery apparatus in the radially-compressed configuration. The method further comprises advancing the prosthetic heart valve to a desired implantation site. The method also comprises using the delivery apparatus to expand the prosthetic heart valve to the radially-expanded configuration, thereby implanting the prosthetic heart valve at the desired implantation site.

In another representative example, a method is provided for implanting in a patient's body the prosthetic heart valve according to any of the above-described representative examples. The method comprises inserting a distal end of a delivery apparatus into vasculature of a patient. The delivery apparatus comprises an elongated shaft. The prosthetic heart valve is releasably supported within the delivery apparatus in the radially-compressed configuration. The method further comprises advancing the prosthetic heart valve to a desired implantation site. The method also comprises deploying the prosthetic heart valve from the delivery apparatus such that the prosthetic heart valve self-expands to a previously shape-set configuration that is intermediate between the radially-compressed and radially expanded configurations. The method further comprises using the delivery apparatus to further expand the prosthetic heart valve to the radially-expanded configuration, thereby implanting the prosthetic heart valve at the desired implantation site.

In another representative example, a method is provided for fabricating a prosthetic heart valve. The method comprises forming a frame having a plurality of first cells. Each first cell comprises a plurality of interconnected first struts and an inner cell disposed within an area bounded by the first struts of the first cell. Each inner cell comprises a plurality of interconnected second struts, a first locking member at an end of a support arm, and a second locking member. The support arm extends from a first end of the respective inner cell toward a second end of the respective inner cell that is opposite the first end. The second locking member is at or adjacent to the second end of the respective inner cell. The frame is formed of a shape memory material. Each first locking member is in an initial orientation. The method further comprises shape-setting each first locking member to have a twisted orientation with respect to the corresponding inner cell.

In another representative example, a method is provided for fabricating a prosthetic heart valve. The method comprises forming a frame having a plurality of first cells and a plurality of first locking members. Each first cell comprises a plurality of interconnected first struts and a second locking member at or adjacent to the second end of the respective first cell. Each first locking member is at an end of a support arm that extends from a first end of a corresponding first cell. The frame is formed of a shape memory material. Each first locking member is in an initial orientation. The method further comprises shape-setting each first locking member to have a twisted orientation with respect to the corresponding first cell.

In another representative example, a method is provided for fabricating a prosthetic heart valve. The method comprises forming a frame having a plurality of first cells. Each first cell comprises a plurality of interconnected first struts, a first locking member at an end of a first support arm, and a second locking member at an end of a second support. The first support arm extends from a first end of the respective first cell toward a second end of the respective first cell that is opposite the first end. The second support arm extends axially from the second end of the respective first cell toward the first end of the respective first cell. The frame is formed of a shape memory material. Each of the first and second locking members is in an initial orientation. The method further comprises shape-setting each of the first and second locking members to have a twisted orientation with respect to the corresponding first cell.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of an exemplary prosthetic heart valve, according to one or more implementations of the disclosed subject matter.

FIG. 2A is an elevation view of a pair of cells from the frame of the prosthetic heart valve of FIG. 1 in an initial configuration.

FIGS. 2B-2C are elevation views of the pair of cells of FIG. 2A in a radially-compressed configuration and in a radially-expanded configuration, respectively.

FIG. 3A is an elevation view of a single cell from the frame of the prosthetic heart valve of FIG. 1 prior to twisting of a female ratchet member.

FIG. 3B is an elevation view of the single cell of FIG. 3A after twisting of the female ratchet member.

FIG. 3C is a perspective view of the single cell of FIG. 3B illustrating locking by male and female ratchet members when the corresponding prosthetic heart valve frame is in the radially-expanded configuration.

FIG. 4A is an elevation view of a single cell from a frame of another exemplary prosthetic heart valve prior to twisting of a female ratchet member, according to one or more implementations of the disclosed subject matter.

FIG. 4B is an elevation view of the single cell of FIG. 4A after twisting of the female ratchet member.

FIG. 4C is a perspective view of the single cell of FIG. 4B illustrating locking by male and female ratchet members when the corresponding prosthetic heart valve frame is in the radially-expanded configuration.

FIG. 4D is an elevation view of a variation for fabricating the single cell of FIG. 4A, according to one or more implementations of the disclosed subject matter.

FIG. 5A is an elevation view of a single cell from a frame of another exemplary prosthetic heart valve prior to twisting of a female ratchet member, according to one or more implementations of the disclosed subject matter.

FIG. 5B is an elevation view of the single cell of FIG. 5A after twisting of the female ratchet member.

FIG. 5C is a perspective view of the single cell of FIG. 5B illustrating locking by male and female ratchet members when the corresponding prosthetic heart valve frame is in the radially-expanded configuration.

FIG. 6A is an elevation view of a single cell from a frame of another exemplary prosthetic heart valve prior to twisting of male and female ratchet members, according to one or more implementations of the disclosed subject matter.

FIG. 6B is an elevation view of the single cell of FIG. 6A after twisting of the male and female ratchet members.

FIG. 6C is a perspective view of the single cell of FIG. 5B illustrating locking by the male and female ratchet members when the corresponding prosthetic heart valve frame is in the radially-expanded configuration.

FIG. 7A is a side view of the frame of the exemplary prosthetic heart valve of FIG. 1 employing a first actuation mechanism for each cell, according to one or more implementations of the disclosed subject matter.

FIG. 7B is a close-up side view of a single cell from the frame of the exemplary prosthetic heart valve of FIG. 1 employing a second actuation mechanism, according to one or more implementations of the disclosed subject matter.

FIG. 7C is a close-up side view of the single cell of FIG. 7B illustrating removal of the second actuation mechanism from the frame.

FIG. 8 is a side view of the frame of another exemplary prosthetic heart valve having an alternating arrangement of actuation mechanisms and locking mechanisms.

FIG. 9A shows an exemplary delivery apparatus that can be used for implanting a prosthetic heart valve, according to one or more implementations of the disclosed subject matter.

FIG. 9B shows a detailed view of the prosthetic heart valve coupled to the delivery apparatus of FIG. 9A.

FIGS. 10A-10E depict stages of an exemplary procedure for implanting a prosthetic heart valve within the native aortic valve of a patient's heart, according to one or more implementations of the disclosed subject matter.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present, or problems be solved. The technologies from any example can be combined with the technologies described in any one or more of the other examples.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed embodiments are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and or.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part, and the object remains the same.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximate unless the word “about” is recited. Whenever “substantially,” “approximately,” “about,” or similar language is explicitly used in combination with a specific value, variations up to and including 10% of that value are intended, unless explicitly stated otherwise.

### Overview of the Disclosed Technology

Prosthetic heart valves disclosed herein can be radially compressed and/or expanded, as well as locked in place by a ratcheting locking mechanism. As one example, the prosthetic heart valves can be crimped on or retained by an implant delivery apparatus in a radially-compressed configuration (also referred to herein as a radially-compressed state or crimped state) during delivery, and then radially expanded (and axially shortened) to a radially-expanded state configuration (also referred to therein a radially-expanded state or deployed state) once the prosthetic heart valve reaches a desired implantation site. The ratcheting locking mechanism can be constructed to hold the prosthetic heart valve in the radially-expanded configuration, so as to prevent the prosthetic heart valve from collapsing after implantation. It should be understood that the prosthetic heart valves disclosed herein may be used with any of a variety of implant delivery apparatuses, and examples thereof will be discussed in more detail hereinbelow.

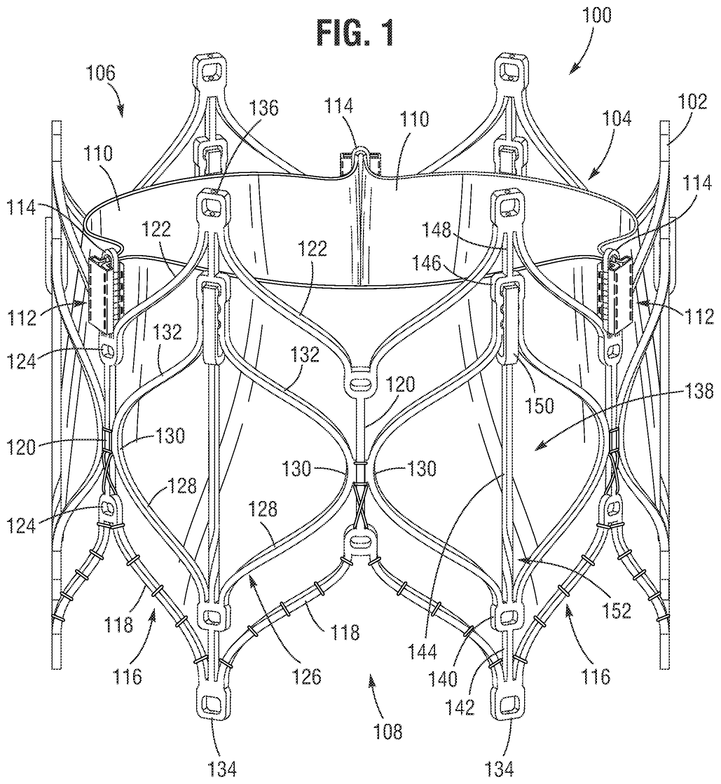

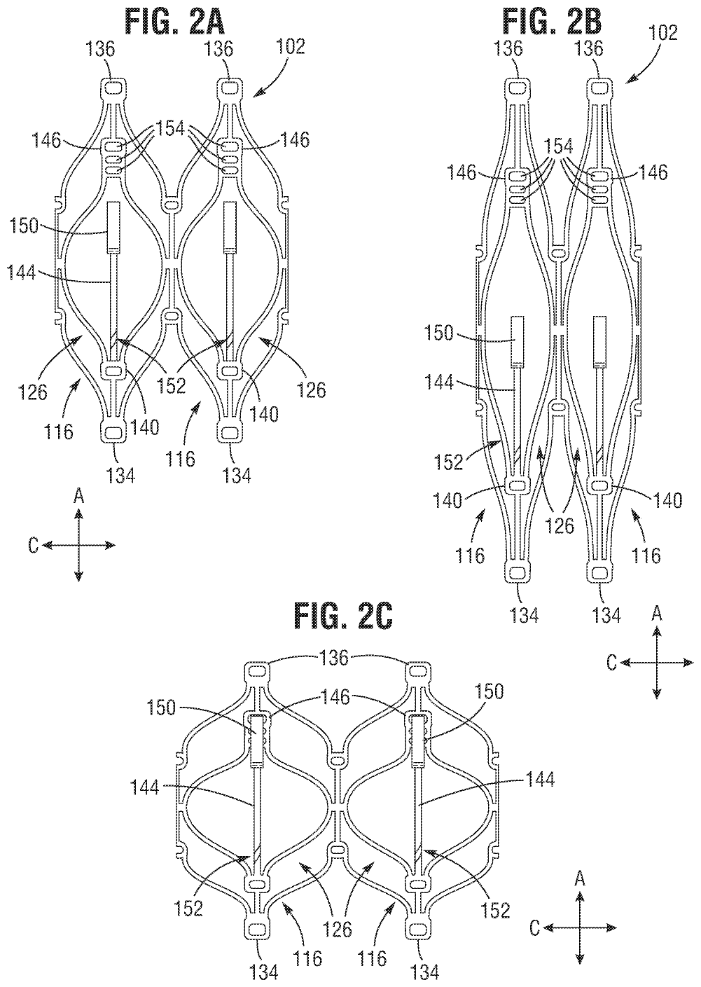

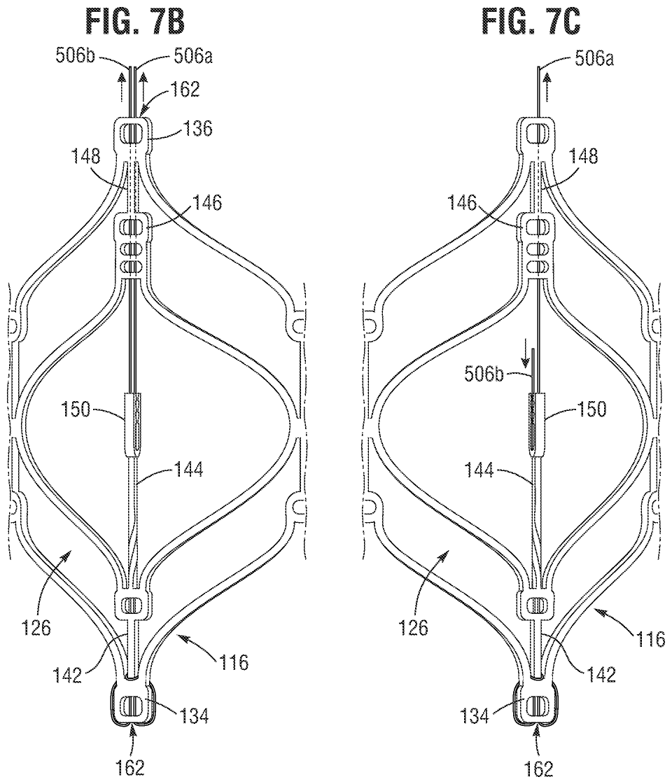

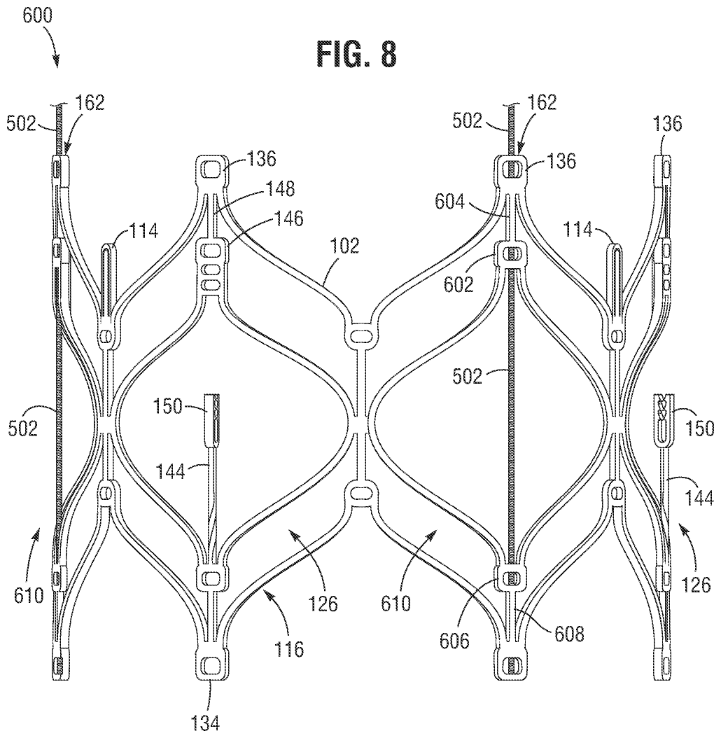

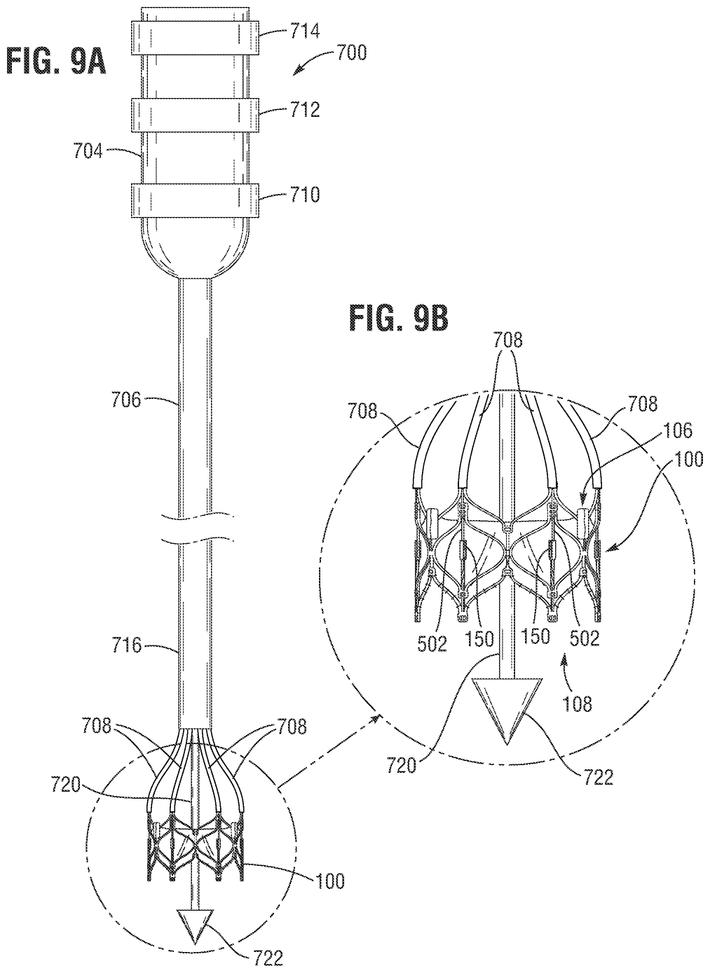

FIG. 1 shows an exemplary prosthetic valve that may be delivered to, and implanted at, a native heart valve by a delivery apparatus, such as the exemplary delivery apparatus shown in FIG. 9A. FIGS. 2A-6C show various examples of ratcheting locking mechanisms that may be included with a prosthetic heart valve (such as the exemplary prosthetic heart valve shown in FIG. 1) to prevent the prosthetic heart valve from collapsing back to a more radially compressed configuration during and/or after radial expansion of the prosthetic heart valve, such as during and/or after implantation of the prosthetic heart valve at a native heart valve or within a previously installed prosthetic valve (e.g., a valve-in-valve procedure). Moreover, FIGS. 7A-8 show various examples of actuation mechanisms in combination with the ratcheting locking mechanisms. The actuation mechanisms can interact with the prosthetic heart valve to cause expansion of the prosthetic heart valve to the radially-expanded configuration.

In some implementations, one or more cells of a frame of the prosthetic heart valve include a ratcheting locking mechanism having first and second locking members. When the frame is initially fabricated, the first locking member can be oriented coplanar with a plane of the corresponding cell. However, any deflection of the first locking member radially inward or outward in this initial configuration would misalign the first locking member with its corresponding second locking member, thereby compromising the ability of the locking members to properly engage with each other upon expansion of the prosthetic heart valve. Accordingly, in some implementations, the first locking member is twisted so as to have an orientation crossing a plane of the cell, for example, perpendicular to the plane of the cell. In this twisted orientation, even if the first locking member deflects to some degree in the radial direction, the first locking member is still able to engage with the second locking member.

For example, the first locking member can be a female ratchet member having teeth on separate jaws designed to engage corresponding openings or recesses of a male ratchet member of the ratcheting locking mechanism. Before the twisting, the teeth of the female ratchet member face toward each other along a direction coplanar with the cell and/or along a direction parallel to the circumferential direction of the frame. After the twisting, the teeth of the female ratchet member face each other along a direction perpendicular to a plane of the cell and/or along a direction parallel to the radial direction of the frame.

### Examples of the Disclosed Technology

FIG. 1 illustrates an exemplary prosthetic heart valve 100 that comprises a frame 102 and a valvular structure 104 supported by the frame 102. The frame 102 (also referred to as a stent) has a first axial end 106 and an opposite second axial end 108. In some implementations, the second axial end 108 serves as an inflow end of the valve 100, while the first axial end 106 serves as an outflow end. In the illustrated example, the frame 102 has a cylindrical or substantially cylindrical shape, with a constant diameter from the inflow end 108 to the outflow end 106, e.g., a substantially annular frame. Alternatively, in some implementations, the frame can have a diameter that varies along the height of the frame (e.g., in a direction from the inflow end to the outflow end), e.g., a tapered frame, for example, as disclosed in U.S. Patent Application Publication Nos. 2012/0239142, 2020/0188099, 2020/0390547, all of which are incorporated herein by reference.

The valvular structure 104 is configured to regulate the flow of blood through the prosthetic valve 100 from the inflow end 108 to the outflow end 106 and comprises a plurality of flexible leaflets 110. For example, each leaflet 110 can be made from in whole or part, biological material, bio-compatible synthetic materials, or other such materials. In the illustrated example, the valvular structure 104 has three leaflets (e.g., arranged to collapse in a tricuspid arrangement), but fewer or additional leaflets are also possible (e.g., two leaflets in a bicuspid arrangement). Each leaflet 110 of the valvular structure 104 can be coupled to the frame 102 at the commissure portions of the frame, in particular by inserting commissure tabs 112 through respective windows 114 and securing thereto. The leaflets 110 can also be coupled to the frame 102 along its inflow edge (e.g., to struts 118 at second axial end 108 of the frame 102, also referred to as “cusp edges”). In some implementations, a reinforcing element or connecting skirt, such as a fabric strip, can be connected directly to the cusp edges of the leaflets and to the struts of the frame to couple the cusp edges of the leaflets to the frame. Alternatively, or additionally, in some implementations, the inflow edge portions of leaflets 110 can be sutured to an inner skirt, which in turn is sutured to the adjacent struts of the frame (e.g., struts 118). Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, and 8,252,202, U.S. Patent Application Publication No. 2018/0325665, and International Application Publication No. WO-2020/198273, all of which are incorporated herein by reference in their entireties.

Although not shown in the illustrated example of FIG. 1, the prosthetic valve 100 can include one or more skirts or sealing members. For example, in some implementations, the prosthetic valve 100 can include an inner skirt (not shown) mounted on the inner surface of the frame. The inner skirt can function as a sealing member to prevent or decrease perivalvular leakage, to anchor the leaflets to the frame, and/or to protect the leaflets against damage caused by contact with the frame during crimping and during working cycles of the prosthetic valve. In some implementations, the valve 100 can also comprise an outer skirt (also referred to as paravalvular sealing member) that is secured to the frame 102 via sutures. Such an outer skirt can comprise a fabric material, tissue material, or similar flexible and flow-proof material, and can help seal the region between the outside of the frame and the native tissue against which the valve is implanted to prevent paravalvular blood flow. The outer skirt can also promote tissue ingrowth and help secure the valve to the tissue. The inner and outer skirts can be formed from any of various suitable biocompatible materials, including any of various synthetic materials, including fabrics (e.g., polyethylene terephthalate fabric) or natural tissue (e.g., pericardial tissue). Further details regarding frame construction, inner and outer skirts, techniques for assembling leaflets to the inner skirt, and techniques for assembling skirts to the frame are disclosed in U.S. Pat. No. 9,393,110, U.S. Patent Application Publication Nos. 2018/0325665, 2019/0105153, and 2019/0365530, and International Publication Nos. WO-2020/159783 and WO-2020/198273, all of which are incorporated herein by reference. In some implementations, the outer skirt can cover most or all of an outer circumferential surface of the frame 102. Further details regarding prosthetic heart valves with outer skirts that cover the valve frame are described in U.S. Patent Application Publication Nos. 2019/0046314, 2019/0192296, and 2019/0374337, all of which are incorporated herein by reference.

The prosthetic valve 100 can be radially compressible and expandable between a radially-compressed configuration (also referred to as the “compressed configuration” or “crimped state”) and a radially-expanded configuration (also referred to as the “expanded configuration” or “implanted state”). The frame 102 can include a plurality of interconnected struts that define respective cells forming a circumferential wall of the frame 102. For example, the wall of the frame 102 can comprise a plurality of outer cells 116 arranged in a single row along a circumferential direction (C) of the frame 102. In the illustrated example, each outer cell 116 is formed by a pair of bottom angled struts 118, a pair of top angled struts 122, and a pair of vertical struts 120. First ends of the bottom angled struts 118 are connected together at lower junction 134 (also referred to herein as a union or junction member) to form an apex at the inflow end, and first ends of the top angled struts 122 are connected together at upper junction 136 to form an apex at the outflow end 106. Opposite second ends of each of the bottom angled struts 118 and the top angled struts 122 are connected to respective vertical struts 120 at junctions 124, with outer cells 116 adjacent in the circumferential direction sharing vertical struts 120 and junctions 124. Struts 118, 120, and 122 thus delineate a border of each outer cell 116 surrounding an inner area.

In some implementations, the frame 102 further comprises a plurality of inner cells 126, each inner cell 126 being disposed within the inner area of a respective outer cell 116. In the illustrated example, each inner cell 126 is formed by a pair of bottom angled struts 128 and a pair of top angled struts 132. First ends of the bottom angled struts 128 are connected together at a lower junction 140, which is in turn connected to the lower junction 134 of the outer cell 116 by a lower vertical strut 142. First ends of the top angled struts 132 are connected together at an upper junction, which also serves as a locking member 146 (also referred to as a second locking member or male locking member). The locking member 146 is in turn connected to the upper junction 136 of the outer cell 116 by an upper vertical strut 148. Opposite second ends of each of the bottom angled struts 128 and the top angled struts 132 are connected together at side junctions 130, which are in turn connected to respective vertical struts 120 (e.g., at a position along the axial direction of the frame 102 between a midpoint of the vertical strut 120 and the respective junction 124 at the inflow end 108 of the frame 102).

In some implementations, at least some of the cells of the frame include a respective locking mechanism, for example, a ratcheting lock mechanism based on engagement between a pair of locking members. For example, each cell of the frame can have a male locking member coupled thereto at one axial end (e.g., closer to the outflow end 106) and a corresponding female locking member coupled at an opposite axial end (e.g., closer to the inflow end 108). As the frame is expanded radially, the height of the frame shortens such that the axial ends of the cell approach each other, thereby causing the male and female locking members to engage with each other. The male and female locking members can include cooperating features that prevent, or at least resist, disengagement after the locking members initially engage with each other. Once the prosthetic valve has been implanted within a patient, the patient's native anatomy (e.g., the native aortic annulus) may exert radial forces (e.g., inward along radial direction R) against the prosthetic valve that would tend to compress the frame. However, the engagement between the male and female locking members prevents such forces from compressing the frame, thereby ensuring that the frame remains locked in the desired radially-expanded configuration.

In some implementations, at least one of the male and female locking members can be mounted on an axially-extending arm. The axially-extending arm can couple the locking member to the corresponding axial end of the frame cell, for example, to reduce the axial distance that the locking member has to travel in order to engage with the corresponding locking member during expansion of the frame. In such configurations, prior to engagement between the locking members, the axially-extending arm may be susceptible to bending in the radial direction, such that the locking members are no longer coaxially aligned. Since the locking members are no longer in the same plane due to the bending of the arm, the locking members may not properly engage with each other during valve expansion. Accordingly, in some implementations, at least one of the male and female locking members have a twisted orientation, such that the twisted locking member is oriented out of the plane of the frame cell (e.g., orthogonal to the plane of the frame cell). For example, when the locking member has a twisted orientation, a first part of the locking member may be disposed radially inward of a second part of the locking member. Alternatively, or additionally, when the locking member has a twisted orientation, a first part of the locking member may be disposed radially inward of a junction of the support arm with the frame cell, and a second part of the locking member may be disposed radially outward of the junction of the support arm with the frame cell. The twisted orientation of one or both of the locking members can allow the locking mechanism to better accommodate any unintended radial deflection of the corresponding support arm and thereby ensure reliable engagement of the locking members upon expansion of the valve.

In some implementations, the support arm has a free end portion, which comprises one of the male and female locking members, and a fixed end portion coupled to the frame cell (e.g., at lower junction 140). The fixed end portion can have a rotational position that is different (e.g., rotationally offset by 90°±n·360°, where n is an integer) from the rotational position of the free end portion. For example, the rotational position at the free end portion can be out of plane of the corresponding frame cell, such that at least some part of the free end portion is disposed radially inward of the frame cell plane (e.g., a curved plane containing the struts defining the frame cell) and at least another part of the free end portion is disposed radially outward of the frame cell plane. The rotational position at the fixed end portion can be substantially coplanar with the plane of the corresponding frame cell (e.g., with axially-extending side surfaces of the fixed end portion of the support arm 144 being substantially coplanar with or parallel to corresponding side surfaces of the vertical strut 142 of the frame cell).

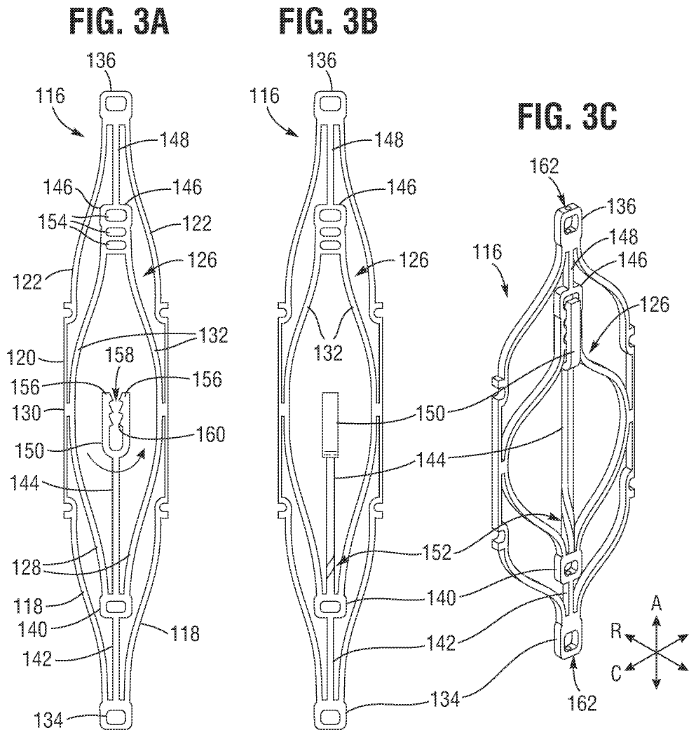

In the illustrated example of FIGS. 1-3C, the female locking member 150 is mounted on an end of the axially-extending support arm 144. The female locking member 150 include a pair of jaws 156 spaced apart from each other by a gap 158 constructed to receive a male locking member therein. Each jaw 156 has a plurality of ratcheting teeth 160 that extend into the gap 158 and toward the opposite jaw 156. The female locking member 150 is twisted from an initial orientation (e.g., coplanar with the frame cell in FIG. 3A) to the twisted orientation (e.g., orthogonal to the frame cell in FIG. 3B). In the illustrated example, the twist 152 to provide the twisted orientation of the female locking member 150 is disposed adjacent to the lower junction 140 of the inner cell 126. However, other locations or configurations for the twist 152 are also possible. For example, in some implementations, the twist may be located adjacent the female locking member 150, or the twist may extend along the length of the support arm between the junction 140 and the female locking member 150. In some implementations, the female locking member itself is twisted in addition to or instead of forming a twist along the support arm.

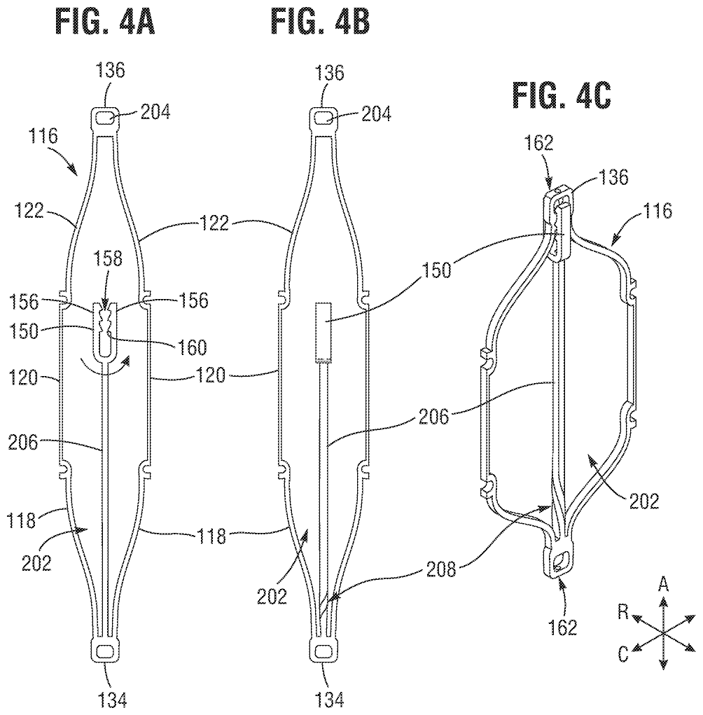

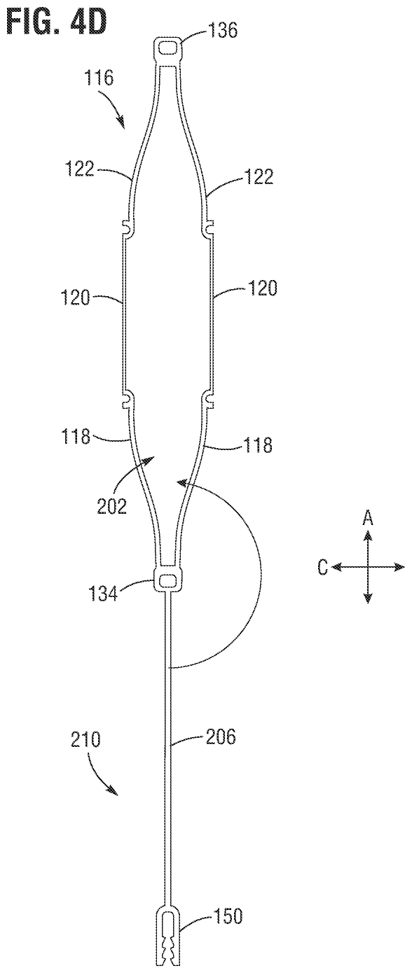

In the illustrated example of FIGS. 1-3C, the male locking member 146 also acts as a junction between the inner cell 126 and the outer cell 116 (e.g., via upper vertical strut 148) and as a junction between top angled struts 132 of the inner cell 126. The male locking member 146 can have a linear array of openings 154 (e.g., through-holes), each opening 154 being constructed to receive respective ones of the ratcheting teeth 160 therein. In some implementations, instead of openings 154, the male locking member 146 can include an array of recesses (also referred to as surface depressions) on one or both faces (e.g., faces perpendicular to the radial direction), with each recess being sized and shaped to receive a respective tooth of the female locking member 150. In some implementations, the male locking member can be mounted on an end of an axially-extending support arm, in addition to or instead of the female locking member being mounted on a support arm. For example, the linear array of openings can instead be provided at the end of support arm 144 and provided with the twisted orientation, while the jaws of the female member can be formed as part of the junction between top angled struts 132. Other variations are also possible according to one or more contemplated, and certain non-limiting examples are discussed further below with respect to FIGS. 4A-6C.

Referring to FIG. 2B, when the frame 102 is in the radially compressed configuration, the female locking member 150 can be spaced from the male locking member 146 along the axial direction (A). As the frame 102 is radially expanded (e.g., by moving the inflow end 108 and the outflow end 106 toward each other), the female locking member 150 and male locking member 146 move closer to each other along the axial direction, as shown in the intermediate configuration of FIG. 2A. As the frame 102 continues to radially expand, the female locking member 150 moves over the male locking member 146, with a first of the ratcheting teeth 160 (e.g., the tooth closest to the outflow end 106) being received in a first of the openings 154 (e.g., the opening closest to the inflow end 108). The width of the gap 158 between facing ratcheting teeth 160 of the jaws 156 of the female locking member 150 can be less than a thickness (e.g., along the radial direction, R, of the frame 102) of the male locking member 146, such that the teeth 160 are resiliently retained in position engaging one of the openings 154 of the male locking member 146 (which can be referred to as an engaged position of the teeth). Each tooth 160 can be shaped with a tapered leading edge and a sharp trailing edge. The tapered leading edge can allow the jaws 156 of the female locking member 150 to deflect outward (e.g., away from each other along the radial direction) as the male locking member 146 is inserted into gap 158, until the tooth 160 is received in a corresponding opening 154. Once the tooth 160 is received in an opening 154, the sharp trailing edge can abut an outflow-end edge of the opening 154 to restrain movement of the female locking member 150 along the axial direction away from the male locking member 146.

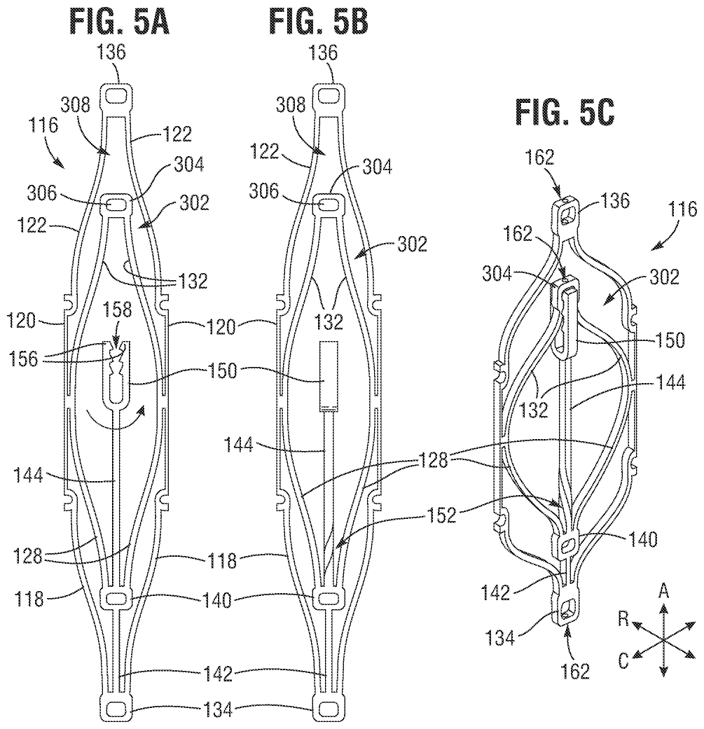

In the illustrated example of FIGS. 1-3C, the male locking member 146 includes an array of multiple openings 154 arranged in sequence along the axial direction, A. Alternatively, in some implementations, the male locking member can include a single opening, such as opening 306 in upper junction 304 of the inner cell 302, as illustrated in the example of FIGS. The frame can be radially expanded until a desired prosthetic valve diameter is achieved, with the desired diameter (e.g., a fully expanded diameter) corresponding to a position of the female locking member 150 with respect to the upper junction 304 where a first of the ratcheting teeth 160 (e.g., the tooth closest to the outflow end 106) can be received in opening 204 when the valve is in the radially-expanded configuration, for example, as shown in FIG. 5C. Alternatively, or additionally, the frame can continue to be expanded such that female locking member extends beyond the upper junction 304, with one or more of the teeth being disposed above the upper junction 304 (e.g., proximal to the inner cell 302) and at least one pair of teeth disposed within the opening 306. With a single opening for the male locking member, the presence of a strut between upper junction of the outer cell and the upper junction of the inner cell may otherwise prevent or impair engagement of the female locking member with the male locking member, for example, due to contact of teeth 160 with the upper strut 148 holding the jaws 156 of the female locking member 150 apart such that other teeth 160 of the jaws are held outside of the opening of the male locking member. Thus, the region 308 (e.g., open area) between the upper junction 136 of the outer cell 116 and the upper junction 304 of the inner cell 302 can be left open (e.g., without an upper strut connecting the inner and outer cells, or at least without an upper strut coaxial with the support arm 144), as illustrated in the example of FIGS.

In some implementations, the components of the frame 102 (e.g., struts, junctions, and locking mechanism) can be made of a self-expanding material (e.g., a shape-memory material), such as a nickel titanium alloy (“NiTi”), for example Nitinol. Alternatively, or additionally, in some implementations, portions of the frame can be formed of plastically-expandable material, such as stainless steel or a cobalt chromium alloy, while other portions of the frame (e.g., locking mechanism 138) are formed of a self-expanding material. Further details regarding the construction of the frame and the prosthetic valve are described in U.S. Patent Application Publication Nos. 2018/0153689, 2018/0344456, 2019/0060057, 2020/0188099, and 2020/0390547, International Application Publication No. WO-2020/081893, and U.S. Patent Application Nos. 63/049,812 filed Jul. 9, 2020, 63/073,622 filed Sep. 2, 2020, and 63/138,599 filed Jan. 18, 2021, all of which are incorporated herein by reference.

In some implementations, when the frame is constructed of a self-expanding material, the frame 102 (and thus the prosthetic heart valve 100) can be crimped to the radially-compressed configuration and restrained in the compressed configuration by insertion into a sheath or equivalent mechanism of a delivery catheter. Once maneuvered to the implantation site, the prosthetic heart valve can be advanced from the delivery sheath, thereby allowing the prosthetic heart valve to expand to its memorized diameter, which may be less than the radially-expanded configuration. For example, in some implementations, the memorized configuration (e.g., as shown in FIG. 2A) may be intermediate between the radially-compressed configuration (e.g., as shown in FIG. 2B) and the radially-expanded configuration (e.g., as shown in FIG. 2C). For example, the memorized configuration may correspond to a valve frame diameter of about mm, while the radially-compressed configuration may correspond to a valve frame diameter approaching 5 mm (e.g., 6-8 mm, inclusive) and the radially-expanded configuration may correspond to a valve frame diameter approaching 30 mm (e.g., 20-30 mm, inclusive). The prosthetic valve 100 can then be further expanded to its full radially-expanded configuration for implantation using, for example, by using an inflatable balloon or equivalent expansion mechanism or by mechanical actuators coupled to the frame 102.

In some implementations, the frame can initially be formed in a flat configuration (e.g., a strip of cells), and then opposite ends thereof coupled together to form the generally annular shape of the frame 102. Alternatively, or additionally, in some implementations, the various structures of the frame (e.g., struts, junctions, and locking mechanisms) can be formed as unitary structure from a single cylindrical member (e.g., hypotube) or from a single flat panel, for example, by laser cutting or other high precision machining technique (e.g., electrical discharge machining, waterjet cutting, etc.). Alternatively, in some implementations, the frame 102 can be constructed by laser welding of individual metal wires or members together, for example, where, for each outer cell 116, a first wire forms one of the top angled struts 122, one of the vertical struts 120, and one of the bottom angled struts 118, and a second wire forms the other of the top angled struts 122, the other of the vertical struts 120, and the other of the bottom angled struts 118.

The frame can initially be formed with both the male locking member and the female locking member being coplanar with the plane of the frame cell. For example, FIG. 3A illustrates a single cell 116 of the frame 102 in the initial as-fabricated configuration (e.g., after cutting or machining). In the coplanar configuration of FIG. 3A, the teeth 160 of the female locking member 150 are oriented toward each other along the same plane of the cell 116, and the openings 154 extend perpendicular to a plane of the cell 116 through a thickness of the male locking member 146. In the configuration of FIG. 3A, however, the support arm 144 extending from the lower junction 140 is susceptible to bending radially inward or outward (e.g., into or out of the plane of the page in FIG. 3A), which bending would cause the female locking member 150 to be offset from the plane of the outer cell 116 and thereby misaligned from the male locking member 146 when the valve is expanded.

To mitigate the effect of any support arm bending, at least part of the locking mechanism is subjected to shape-setting (e.g., heat-setting) to change a rotational position of one or both of the locking members with respect to the plane of the cell 116. In the illustrated example of FIG. 3B, the support arm 144 and female locking member 150 are rotated by 90° (or ±90°±n·360°), such that the teeth 160 of the female locking member 150 are oriented toward each other along a plane perpendicular to that of the cell 116. For example, in some implementations, the female locking member 150 can be grasped and rotated with respect to the junction 140 of the support arm 144. The rotation causes deformation of the support arm 144 such that a twist 152 is formed therealong. In the illustrated example, the twist 152 is disposed in a region of the support arm 144 adjacent to junction 140. However, in some implementations, the twist 152 can be disposed anywhere along the length of the support arm. In some implementations, the frame 102 can be mounted on a mandrel with holes therein corresponding to the locations of the female locking members. A rod can be used to grasp and rotate the female locking member. Other mechanisms for grasping and rotating parts of the locking mechanism are also possible according to one or more contemplated implementations.

To provide the shape-setting, the frame 102 is held at a temperature in excess of a transition temperature of its constituent shape-memory material during the rotation. Alternatively, in some implementations, the portion of the frame 102 being shaped (e.g., the female locking member and support arm) can be locally heated above the transition temperature. In either case, the frame 102 can then be cooled to a temperature below the transition temperature to set the current shape as the original pre-deformed shape. In some implementations, the female locking member 150 and support arm 144 are rotated in a single step to the final rotational position of FIG. 3B. Alternatively, in some implementations, the rotation to the final rotational position of FIG. 3B is accomplished by a series of incremental steps, each comprising heating above the transition temperature, rotating an incremental amount (e.g., 10-30°), and cooling below the transition temperature. Deformation of the frame 102 while below the transition temperature (e.g., in transitioning to the compressed configuration) can be effectively undone by heating to a temperature about the transition temperature, whereby the frame 102 automatically reverts to its original pre-deformed shape. Further details regarding shape memory material fabrication techniques, which can be used to form frame 102, can be found in U.S. Pat. Nos. 5,540,712 and 8,187,396, each of which is incorporated herein by reference.

By adopting the twisted configuration for the female locking member 150, even if the support arm deviates to some degree in the radial direction, the male locking member 146 can still slide over the teeth 160 of the female locking member 150 during expansion of the valve and properly engage therewith, as illustrated in FIG. 3C. It should be noted that the likelihood of lateral displacement (e.g., along the circumferential direction) of the support arm 144, which would lead to misalignment between the teeth 160 of the female locking member 150 and openings 154 of the male locking member 146, may be substantially less than the likelihood of radial displacement of the support arm 144 due to the surrounding structures of the valve frame, for example, the bottom angled struts 128 connecting to lower junction 140. Accordingly, the transition to the rotational position of FIG. 3B decreases the overall risk of misalignment between the female locking member 150 and the male locking member 146, and ensures proper engagement of the locking mechanism within each cell during expansion of the frame 102. In some implementations, additional alignment and guidance between the female locking member 150 and the male locking member 146 can be provided by an actuator (e.g., a threaded rod) inserted into actuator lumen 162 (also referred to as an actuator conduit) and passing through each of the locking members. Although only a single cell is shown in the illustrated example of FIGS. 3A-3C, it will be appreciated that other cells 116 of the frame 102 that are arrayed in a single row around the circumference of the frame 102 would have a similar configuration and be fabricated in a similar manner.

In some implementations, the inner cells can be omitted from the frame of the prosthetic heart valve, with at least some of the outer cells of the frame including a respective ratcheting locking mechanism. For example, FIGS. 4A-4C illustrates a single cell 116 of a frame where the internal area 202 of the cell 116 lacks a corresponding inner cell (e.g., inner cell 126). Similar to the example of FIGS. 1-3C, the female locking member 150 in the example of FIGS. 4A-4C is mounted on an end of an axially-extending support arm 206; however, the support arm 206 extends from lower junction 134. The female locking member 150 include a pair of jaws 156 spaced apart from each other by a gap 158 constructed to receive a male locking member therein. Each jaw 156 has a plurality of ratcheting teeth 160 that extend into the gap 158 and toward the opposite jaw 156.

The female locking member 150 is twisted from an initial orientation (e.g., coplanar with the frame cell in FIG. 4A) to the twisted orientation (e.g., orthogonal to the frame cell in FIG. 4B), for example, via a shape-setting technique similar to that described above with respect to FIGS. 3A-3C. In the illustrated example, the twist 208 to provide the twisted orientation of the female locking member 150 is disposed adjacent to the lower junction 134 of the outer cell 116. However, other locations or configurations for the twist 208 are also possible. For example, in some implementations, the twist may be located adjacent the female locking member 150, or the twist may extend along the length of the support arm 206 between the junction 134 and the female locking member 150. In some implementations, the female locking member itself is twisted in addition to or instead of forming a twist along the support arm.

In the illustrated example of FIGS. 4A-4C, the upper junction 136 between top angled struts 122 of the outer cell 116 also acts as the male locking member. The upper junction 136 has a single opening 204 (e.g., through-holes) constructed to receive ones of the ratcheting teeth 160 therein. Alternatively, in some implementations, the upper junction 136 can have an array of multiple openings, similar to that illustrated in FIGS. 1-3C. As the frame is expanded (e.g., by moving the inflow end 108 and the outflow end 106 toward each other) the female locking member 150 and upper junction 136 move closer to each other along the axial direction, until the female locking member 150 engages with the upper junction 136 in the radially-expanded configuration. In the illustrated example of FIG. 4C, a first of the ratcheting teeth 160 (e.g., the tooth closest to the outflow end 106) can be received in opening 204 when the valve is in the radially-expanded configuration. Alternatively, or additionally, the frame can continue to be expanded such that female locking member extends beyond the upper junction 136, with one or more of the teeth being disposed above the upper junction 136 (e.g., proximal to the frame) and at least one pair of teeth disposed within the opening 204.

The width of the gap 158 between facing ratcheting teeth 160 of the jaws 156 of the female locking member 150 can be less than a thickness (e.g., along the radial direction, R, of the frame 102) of the upper junction 146, such that teeth 160 are resiliently retained in position engaging opening 204 of the upper junction 146 (which can be referred to as an engaged position of the teeth). Each tooth 160 can be shaped with a tapered leading edge and a sharp trailing edge. The tapered leading edge can allow the jaws 156 of the female locking member 150 to deflect outward (e.g., away from each other along the radial direction) as the upper junction 136 is inserted into gap 158, until the tooth 160 is received into opening 204. Once the tooth 160 is received in opening 204, the sharp trailing edge can abut an outflow-end edge of the opening 204 to restrain movement of the female locking member 150 along the axial direction away from the upper junction 136. In some implementations, instead of openings 204, the upper junction 136 can include a recess (also referred to as surface depressions) on one or both faces (e.g., faces perpendicular to the radial direction), with each recess being sized and shaped to receive a respective tooth of the female locking member 150.

The axial distance between lower junction 134 and upper junction 136 of the cell 116 in the crimped state for the example of FIGS. 4A-4C is generally greater than the axial distance between lower junction 140 and the upper junction 146 of the inner cell 126 in the crimped state for the example of FIGS. 3A-3C, but the change in the axial distances due to expansion of the valve frame may generally be the same. Accordingly, the length of support arm 206 along the axial direction in FIGS. 4A-4C is increased as compared to the length of support arm 144 along the axial direction in FIGS. 3A-3C in order to ensure that the female locking member 150 engages with the upper junction 136 upon expansion of the valve frame.

By adopting the twisted configuration for the female locking member 150, even if the support arm 206 deviates to some degree in the radial direction, the upper junction 136 can still slide over the teeth 160 of the female locking member 150 during expansion of the valve and properly engage therewith, as illustrated in FIG. 4C. It should be noted that the likelihood of lateral displacement (e.g., along the circumferential direction) of the support arm 206, which would lead to misalignment between the teeth 160 of the female locking member 150 and opening 204 of the upper junction 136, may be substantially less than the likelihood of radial displacement of the support arm 206 due to the surrounding structures of the valve frame, for example, the bottom angled struts 118 connecting to lower junction 134. Thus, the transition to the rotational position of FIG. 4B decreases the overall risk of misalignment between the female locking member 150 and upper junction 136 and ensures proper engagement of the locking mechanism within each cell during expansion of the frame 102. In some implementations, additional alignment and guidance between the female locking member 150 and the upper junction 136 can be provided by an actuator (e.g., a threaded rod) inserted into actuator lumen 162 and passing through each of the locking members. Although only a single cell is shown in the illustrated example of FIGS. 4A-4C, it will be appreciated that other cells 116 of the frame that are arrayed in a single row around the circumference of the frame would have a similar configuration and be fabricated in a similar manner.

Similar to the examples noted above, the frame in the example of FIGS. 4A-4C can initially be formed in a flat configuration (e.g., a strip of cells), and then opposite ends thereof coupled together to form the generally annular shape of the frame. Alternatively, in some implementations, the various structures of the frame (e.g., struts, junctions, and locking mechanisms) in the example of FIGS. 4A-4C can be formed from a single cylindrical member (e.g., hypotube), for example, by laser cutting or other high precision machining technique (e.g., electrical discharge machining, waterjet cutting, etc.). Alternatively, in some implementations, the frame in the example of FIGS. 4A-4C can be constructed by laser welding of individual metal wires or members together, for example, where, for each outer cell 116, a first wire forms one of the top angled struts 122, one of the vertical struts 120, and one of the bottom angled struts 118, and a second wire forms the other of the top angled struts 122, the other of the vertical struts 120, and the other of the bottom angled struts 118.

In some implementations, one or more of the components of the locking mechanism can initially be formed outside of the frame cell and then rotated into position within the frame cell. For example, FIG. 4D illustrates an initial configuration were the support arm 206 and female locking member 150 are disposed in a distal region 210 outside the internal area 202 of cell 116, in particular, below lower junction 134. The support arm 206 can then be rotated upward about lower junction 134 (e.g., 180° about an axis parallel to the circumferential direction, C), such that the support arm 206 and female locking member 150 are disposed within internal area 202, similar to the configuration illustrated in FIGS. 4A-4B. The rotation of the support arm 206 can be performed using a shape-setting technique (e.g., incrementally, with multiple heating and cooling cycles) similar to that described above with respect to FIGS. 3A-3C. In some implementations, the female locking member 150 and/or support arm 206 can be provided with the twisted orientation prior to being rotated from distal region 210 into the frame cell 116. In such implementations, the initial location of the female locking member 150 and the support arm 206 outside the internal area 202 of the frame cell 116 may offer improved access for grasping the female locking member 150 to effect said twisting and/or may allow potential high stress or high strain regions due to the twisting to be moved to more favorable locations.

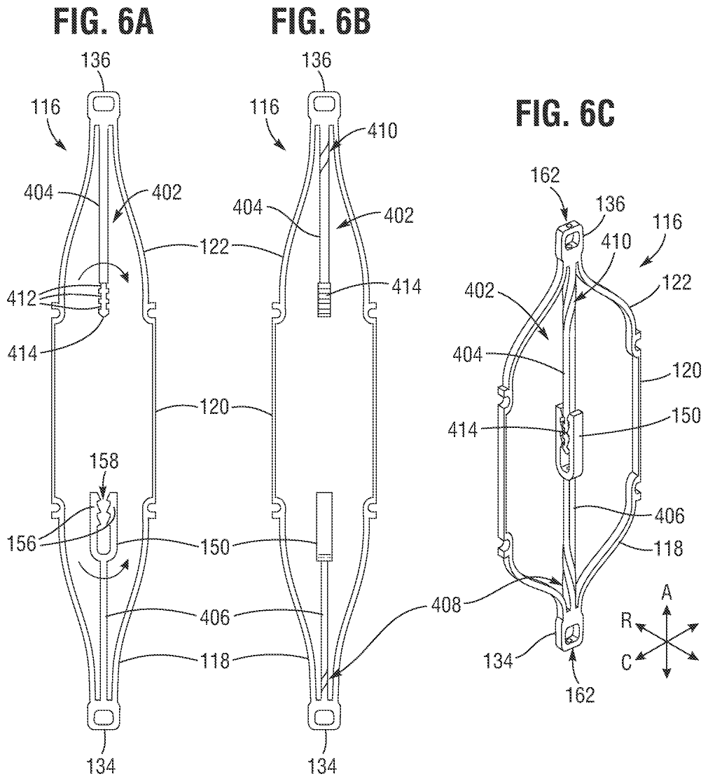

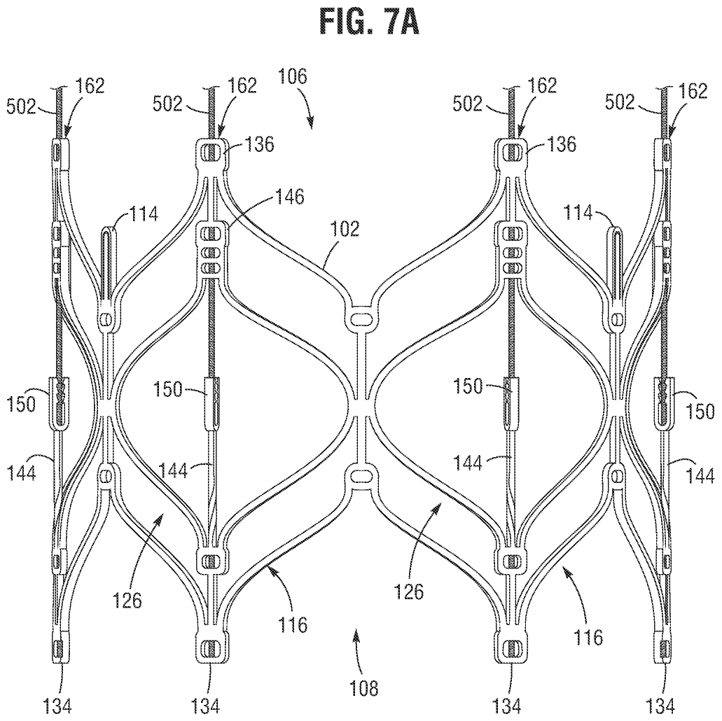

In some implementations, both the female and male locking members can be supported at the end of respective support arms. For example, male locking member 146 with the array of openings 154 can be supported on a support arm that extends from an upper junction of inner cell 126, in a manner similar to female locking member 150 and support arm 144. However, one of the male and female locking members may retain its initial orientation coplanar with the frame cell, while the other of the male and female locking members may be twisted to the twisted orientation (e.g., orthogonal to the frame cell). Alternatively, in some implementations, the openings of the male locking member can be replaced with recessed portions, for example, recessed portions 412 as shown in FIGS. 6A-6C. In the illustrated example, both the male locking member 414 and the female locking member 150 are disposed within the internal area 402 of the frame cell 116 without any inner cell. The female locking member 150 is mounted at the end of support arm 406 that extends from lower junction 134 toward one end of the valve, and the male locking member 414 is mounted at the end of another support arm 404 from upper junction 136 toward an opposite end of the valve.

Similar to the above-described examples, the frame can initially be formed with both the male locking member 414 and the female locking member 150 being coplanar with the plane of the frame cell 116, as shown in FIG. 6A. In the coplanar configuration of FIG. 6A, a depth of each recessed portion 412 is along the plane of the cell. In the configuration of FIG. 6A, however, both support arms 404, 406 may be susceptible to bending radially inward or outward (e.g., into or out of the plane of the page in FIG. 6A), which bending would cause the respective locking member to be offset from the plane of the outer cell 116 and thereby misaligned from the other locking member when the valve is expanded. To mitigate the effect of any support arm bending, both the male locking member 414 (and/or support arm 404) and the female locking member 150 (and/or support arm 406) can be subjected to shape-setting (e.g., heat-setting) to change a rotational position thereof with respect to the plane of the cell 116.

The male locking member 414 and the female locking member 150 can each be twisted from an initial orientation (e.g., coplanar with the frame cell in FIG. 6A) to the twisted orientation (e.g., orthogonal to the frame cell in FIG. 6B), for example, via a shape-setting technique similar to that described above with respect to FIGS. 3A-3C. In the illustrated example, the twist 408 to provide the twisted orientation of the female locking member 150 is disposed adjacent to the lower junction 134 of the frame cell 116, and the twist 410 to provide the twisted orientation of the male locking member 414 is disposed adjacent to the upper junction 136 of the frame cell 116. However, other locations or configurations for the twist 408 and twist 410 are also possible. For example, in some implementations, the twist may be located adjacent the respective locking member, or the twist may extend along the length of the support arm between the respective junction and locking member.

The width of the gap 158 between facing ratcheting teeth 160 of the jaws 156 of the female locking member 150 can be less than a thickness (e.g., between opposite surfaces along the circumferential direction, C, of the frame 102 in FIG. 6A, and along the radial direction, R, of the frame 102 in FIG. 6B) of the male locking member outside of the recessed portions 412, such that teeth 160 are resiliently retained in position engaging recessed portions 412 of the upper junction 146 (which can be referred to as an engaged position of the teeth). As describe above, each tooth 160 can be shaped with a tapered leading edge and a sharp trailing edge, such that the jaws 156 of the female locking member 150 deflect outward (e.g., away from each other along the radial direction) as the male locking member 414 is inserted into gap 158, until a tooth 160 is received into recessed portion 412. Once the tooth 160 is received in recessed portion 412, the sharp trailing edge can abut an outflow-end edge of the recessed portion 412 to restrain movement of the female locking member 150 along the axial direction away from male locking member 414. The shape of the teeth 160 and/or the recessed portions 412 can thus allow the axial ends of the valve frame to move toward each other, but restrict movement of the axial ends of the valve frame away from each other once the locking members engage.

The axial distance between lower junction 134 and upper junction 136 of the cell 116 in the crimped state for the example of FIGS. 6A-6C is generally greater than the axial distance between lower junction 140 and the upper junction 146 of the inner cell 126 in the crimped state for the example of FIGS. 3A-3C, but the change in the axial distances due to expansion of the valve frame may generally be the same. Accordingly, the lengths of support arm 206 and support arm 404 along the axial direction in FIGS. 6A-6C can be selected to ensure that the female locking member 150 engages with the male locking member 414 upon expansion of the valve frame.

By adopting the twisted configuration for both the male locking member 414 and the female locking member 150, even if one or both of support arms 404, 406 deviates to some degree in the radial direction, the male locking member 414 can still slide over the teeth 160 of the female locking member 150 during expansion of the valve and properly engage therewith, as illustrated in FIG. 6C. It should be noted that the likelihood of lateral displacement (e.g., along the circumferential direction) of the support arms 404, 406, which would lead to misalignment between the female locking member 150 and the male locking member 414, may be substantially less than the likelihood of radial displacement of the support arms 404, 406 due to the surrounding structures of the valve frame, for example, bottom angled struts 118 connecting to lower junction 134 and upper angled struts 122 connecting to upper junction 136. Thus, the transition to the rotational positions of FIG. 6B decreases the overall risk of misalignment between the female locking member 150 and the male locking member 414 and ensures proper engagement of the locking mechanism within each cell during expansion of the frame 102. In some implementations, additional alignment and guidance between the female locking member 150 and the male locking member 414 can be provided by an actuator (e.g., a threaded rod) inserted into actuator lumen 162 and passing through each of the locking members. Although only a single cell is shown in the illustrated example of FIGS. 6A-6C, it will be appreciated that other cells 116 of the frame that are arrayed in a single row around the circumference of the frame would have a similar configuration and be fabricated in a similar manner.

Similar to the examples noted above, the frame in the example of FIGS. 6A-6C can initially be formed in a flat configuration (e.g., a strip of cells), and then opposite ends thereof coupled together to form the generally annular shape of the frame. Alternatively, in some implementations, the various structures of the frame (e.g., struts, junctions, and locking mechanisms) in the example of FIGS. 6A-6C can be formed from a single cylindrical member (e.g., hypotube), for example, by laser cutting or other high precision machining technique (e.g., electrical discharge machining, waterjet cutting, etc.). Alternatively, in some implementations, the frame in the example of FIGS. 6A-6C can be constructed by laser welding of individual metal wires or members together, for example, where, for each outer cell 116, a first wire forms one of the top angled struts 122, one of the vertical struts 120, and a second wire forms one of the bottom angled struts 118, and the other of the top angled struts 122, the other of the vertical struts 120, and the other of the bottom angled struts 118.

In any of the examples described herein, the prosthetic valve can be mechanically expanded from the radially-compressed configuration to the radially-expanded configuration. For example, the prosthetic valve 100 can be radially expanded by maintaining the inflow end 108 of the frame 102 at a fixed position while applying a force in the axial direction against the outflow end 106 toward the inflow end 108. Alternatively, the prosthetic valve 100 can be expanded by applying an axial force against the inflow end 108 (e.g., toward outflow end 106) while maintaining the outflow end 106 at a fixed position, or by simultaneously applying opposing axial forces to both the inflow end 108 and the outflow end 106 (e.g., urging the ends toward each other). In some implementations, the axial forces to mechanically expand the valve can be provided by one or more actuators releasably coupled to the frame 102, for example, by insertion into actuator lumens 162 extending through portions of the frame. For example, in some implementations, a plurality of actuators of a delivery apparatus are equally spaced around the circumference of the frame 102 and coupled thereto.

In the illustrated example of FIG. 7A, expansion forces can be applied to the frame 102 by actuators, each of which comprising a screw or threaded rod 502 (also referred to as rotatable actuation member or actuation rod) inserted into an axially-extending lumen of the frame. The rod 502 can extend through various structures of the frame, including, for example, the upper junction 136, upper vertical strut 148, the male locking member 146, the female locking member 150, the support arm 144, the lower junction 140 of the inner cell 126, the lower vertical strut 142, and the lower junction 134 of the outer cell 116. Only four actuator rods 502 are shown in the example of FIG. 7A, although a greater or fewer number of actuators could be used. For example, each cell 116 of the frame 102 can have an associated actuator rod 502 (e.g., six actuator rods corresponding to six frame cells).

Alternatively, in some implementations, only some, but not all, of the cells 116 of the frame can be provided with an actuator rod. Alternatively, or additionally, in some implementations, only some, but not all, of the cells 116 of the frame can be provided with a locking mechanism. For example, cells of the frame can alternate in the circumferential direction between having locking mechanism without an actuator therein and having an actuator without a locking mechanism, as illustrated in the exemplary configuration 600 of FIG. 8. The frame cells 116 that include only locking mechanisms can have a configuration for the respective inner cell 126 similar to that illustrated in FIGS. 1-3C or similar to the other configurations illustrated in any of FIGS. 4A-6C. The other frame cells 116 that include only actuators 502 can have a configuration for the respective inner cell 610 different than the other inner cells 126. For example, the lower junction 606 and lower vertical arm 608 of inner cell 610 can be similar to that of inner cell 126, but the upper junction 602 and/or upper vertical arm 604 of inner cell 610 can different than that of inner cell 126. Other configurations for the first set of frame cells having locking mechanisms and the second set of frame cells having actuators are also possible according to one or more contemplated implementations.

In another example, each cell 116 of the frame can be provided with an actuator rod 502 (e.g., similar to the configuration in the example of FIG. 7A), but cells can otherwise alternate in the circumferential direction between having locking mechanisms and lacking locking mechanisms (e.g., similar to the configuration in the example of FIG. 8). Other configurations of actuators and locking mechanisms with respect to cells of the frame are also possible according to one or more contemplated implementations. In some implementations, cells that have locking mechanisms can be shape set for a different valve diameter than those cells that do not have locking mechanisms. For examples, cells that have locking mechanisms can be shape set to have a size in the memorized configuration corresponding to a valve diameter of 10 mm, while cells that do not have locking mechanisms can be shape set to have a size in the memorized configuration corresponding to a valve diameter approaching 30 mm.

In some implementations, each actuator rod 502 can be threadedly engaged with the upper junction 136, such that when the screws are rotated the upper junctions 136 move axially along the screws toward the lower junctions 134 to axially shorten the frame and thereby radially expand the frame. The screws can also be rotated the opposite direction to radially collapse the frame. Alternatively, in some implementations, each actuator rod can further include a first anchor (e.g., in the form of a cylinder or sleeve) and a second anchor (e.g., in the form of a threaded nut). The threaded actuator rod can extend through the sleeve and the nut. The sleeve can be secured to the frame, such as with a fastener at the junction between two struts. Each actuator rod 502 can be configured to decrease the distance between the attachment locations of the first and second anchors, which causes the frame to foreshorten axially and expand radially. For example, each rod 502 can have external threads that engage internal threads of the nut such that rotation of the rod causes corresponding axial movement of the nut toward or away from the sleeve (depending on the direction of rotation of the rod). This causes the frame junctions at the sleeve and the nut to move closer towards each other to radially expand the frame or to move farther away from each other to radially compress the frame, depending on the direction of rotation of the rod 502. In other implementations, the actuators can be reciprocating-type actuators configured to apply axial directed forces to the frame to produce radial expansion and compression of the frame. For example, the rod 502 of each actuator can be fixed axially relative to the second anchor and slidable relative to the first anchor. Thus, in this manner, moving the rod 502 distally relative to the first anchor and/or moving the first anchor proximally relative to the rod 502 radially compresses the frame. Conversely, moving the rod 502 proximally relative to the first anchor and/or moving the first anchor distally relative to the rod 502 radially expands the frame.

Alternatively, in some implementations, the actuator can be a tension member (e.g., pull cable, wire, or suture) rather than a threaded rod. For example, an actuation assembly can comprise a support tube (e.g., 708 in FIGS. 9A-9B) and a tension member extending through a lumen 162 of the frame cell 116. A distal end portion of the support tube can engage or abut the upper junction 136 of the frame cell 116 and the tension member can be releasably coupled to the lower junction 134 of the frame cell 116. In some implementations, the tension member can extend out of the lumen 162 at the lower junction 134 and be tied (e.g., cross-knot), crimped, or wrapped around the lower junction 134.

For example, as illustrated in FIG. 7B, tension member 506 can be extend through the lumen 162 from the upper junction 136, through structures of the cell 116, and out through the lower junction 134, where the tension member 506 is wrapped around the lower junction 134 and reinserted back into the lumen 162 to extend from the lower junction 134, through structures of the cell 116, and back out through the upper junction 136 such that end 506b of the tension member extends from the upper junction 136. To apply an axial force to the lower junction 134 so as to move it toward the upper junction 136, both ends 506a, 506b of the tension member are pulled proximally (e.g., using a delivery apparatus), as shown in FIG. 7B. When further actuation is no longer needed (e.g., once the valve frame has been partially or fully expanded), the tension member can be removed, for example, by releasing end 506b and pulling end 506a proximally, thereby allowing end 506b to be pulled through and out of lumen 162. Note that FIG. 7C shows the valve frame in a radially-collapsed configuration for illustration purposes; however, in practical implementations, the tension member 506 would be removed once the valve frame is in the radially-expanded configuration and the female locking member 150 is engaged with the male locking member 146.

Further details and examples of actuators and delivery apparatuses for actuating the actuators can be found in U.S. Pat. Nos. 10,603,165 and 10,806,573, U.S. Patent Application Publication Nos. 2018/0153689, 2018/0311039, 2018/0325665, and 2019/0060057, U.S. Patent Application Nos. 62/990,299, 63/085,947, and 63/138,599, International Application Publication No. WO-2020/102487, and International Application Nos. PCT/US2020/057691 and PCT/US2020/063104, all of which are incorporated by reference. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves disclosed herein.

FIG. 9A illustrates an exemplary delivery apparatus 700 adapted to deliver a prosthetic heart valve, such as prosthetic heart valve 100 or any variation thereof described herein. Actuation members (e.g., threaded rod, sutures, etc.) of the delivery apparatus can be releasably inserted into respective actuator lumens of the valve frame 102, or otherwise coupled thereto. Alternatively, the prosthetic valve 100 can be releasably coupled to the delivery apparatus 700, such as via a removable coupling between a distal member of an integrated actuation member of the prosthetic valve 100 and a second actuation member of an actuation assembly of the delivery apparatus 700. The prosthetic valve 100 can include a distal end 108 and a proximal end 106, wherein the proximal end 106 is positioned closer to a handle 704 of the delivery apparatus 700 than the distal end 108, and wherein the distal end 108 is positioned farther from the handle 704 than the proximal end 106. It should be understood that the delivery apparatus 700 and other delivery apparatuses disclosed in the documents incorporated by reference can be used to implant prosthetic devices other than prosthetic valves, such as stents or grafts.

The delivery apparatus 700 in the illustrated example generally includes the handle 704, a first elongated shaft 706 (which comprises an outer shaft in the illustrated example) extending distally from the handle 704, and at least one actuator assembly 708 extending distally through the outer shaft 706. In some implementations, a distal end portion 716 of the shaft 706 can be sized to house the prosthetic valve in its radially compressed, crimped state during delivery of the prosthetic valve through the patient's vasculature. In this manner, the distal end portion 716 functions as a delivery sheath or capsule for the prosthetic valve during delivery. Alternatively, or additionally, a second shaft 726 (e.g., inner or second sheath) within the distal end portion 716 can house the prosthetic valve in the crimped state for delivery.

The at least one actuator assembly 708 can be configured to radially expand and/or radially collapse the prosthetic valve 100 when actuated, and may be removably coupled to the prosthetic heart valve 100. Although the illustrated example of FIGS. 9A-9B shows four actuator assemblies 708 for purposes of illustration, it should be understood that one actuator assembly 708 can be provided for each outer cell of the prosthetic valve or only some of the outer cells of the prosthetic valve, for example, as described above with respect to FIGS. 7A and 8. For example, six actuator assemblies 708 can be provided for a prosthetic valve having six outer cells. In other implementations, a greater or fewer number of actuator assemblies can be present.

The actuator assemblies 708 can be releasably coupled to the prosthetic valve 100. For example, in the illustrated example, each actuator assembly 708 can be coupled to a respective threaded rod within an actuator lumen of the prosthetic valve 100. Each actuator assembly 708 can comprise a support tube or sleeve and an actuator member. In some implementations, the actuator assembly 708 also can include a locking tool. When actuated, the actuator assembly can transmit pushing and/or pulling forces to portions of the prosthetic valve to radially expand and collapse the prosthetic valve. The actuator assemblies 708 can be at least partially disposed radially within, and extend axially through, one or more lumens of the outer shaft 706. For example, the actuator assemblies 708 can extend through a central lumen of the shaft 706 or through separate respective lumens formed in the shaft 706.

The handle 704 of the delivery apparatus 700 can include one or more control mechanisms (e.g., knobs or other actuating mechanisms) for controlling different components of the delivery apparatus 700 in order to expand and/or deploy the prosthetic valve 100. For example, in the illustrated example the handle 704 comprises a first knob 710, a second knob 712, and a third knob 714. The first knob 710 can be a rotatable knob configured to produce axial movement of the outer shaft 706 relative to the prosthetic valve 100 in the distal and/or proximal directions in order to deploy the prosthetic valve from the distal end portion 716 once the prosthetic valve has been advanced to a location at or adjacent the desired implantation location with the patient's body. For example, rotation of the first knob 710 in a first direction (e.g., clockwise) can retract the distal end portion 716 proximally relative to the prosthetic valve 100 and rotation of the first knob 710 in a second direction (e.g., counter-clockwise) can advance the distal end portion 716 distally. In other implementations, the first knob 710 can be actuated by sliding or moving the knob 710 axially, such as pulling and/or pushing the knob. In other implementations, actuation of the first knob 710 (rotation or sliding movement of the knob 710) can produce axial movement of the actuator assemblies 708 (and therefore the prosthetic valve 100) relative to the distal end portion 716 to advance the prosthetic valve distally from the distal end portion 716.

The second knob 712 can be a rotatable knob configured to produce radial expansion and/or contraction of the prosthetic valve 100. For example, rotation of the second knob 712 can move the actuator member and the support tube axially relative to one another. Rotation of the second knob 712 in a first direction (e.g., clockwise) can radially expand the prosthetic valve 100 and rotation of the second knob 712 in a second direction (e.g., counter-clockwise) can radially collapse the prosthetic valve 100. In other implementations, the second knob 712 can be actuated by sliding or moving the knob 712 axially, such as pulling and/or pushing the knob.

The third knob 714 can be a rotatable knob configured to retain the prosthetic heart valve 100 in its expanded configuration. For example, the third knob 714 can be operatively connected to a proximal end portion of the locking tool of each actuator assembly 708. Rotation of the third knob in a first direction (e.g., clockwise) can rotate each locking tool to advance the locking nuts to their distal positions to resist radial compression of the frame of the prosthetic valve. Rotation of the knob 714 in the opposite direction (e.g., counterclockwise) can rotate each locking tool in the opposite direction to decouple each locking tool from the prosthetic valve 100. In other implementations, the third knob 714 can be actuated by sliding or moving the third knob 714 axially, such as pulling and/or pushing the knob.

Although not shown, in some implementations, the handle 704 can include a fourth rotatable knob operative connected to a proximal end portion of each actuator member. The fourth knob can be configured to rotate each actuator member, upon rotation of the knob, to unscrew each actuator member from the proximal portion of a respective actuator. Once the locking tools and the actuator members are uncoupled from the prosthetic valve 100, they can be removed from the patient. Further details of delivery apparatuses that can be used to deliver and implant self-expandable prosthetic valves (including any of the prosthetic valves disclosed herein when the frames are constructed of a self-expandable material such as nitinol) are disclosed in U.S. Pat. Nos. 8,652,202 and 9,867,700, each of which is incorporated herein by reference.

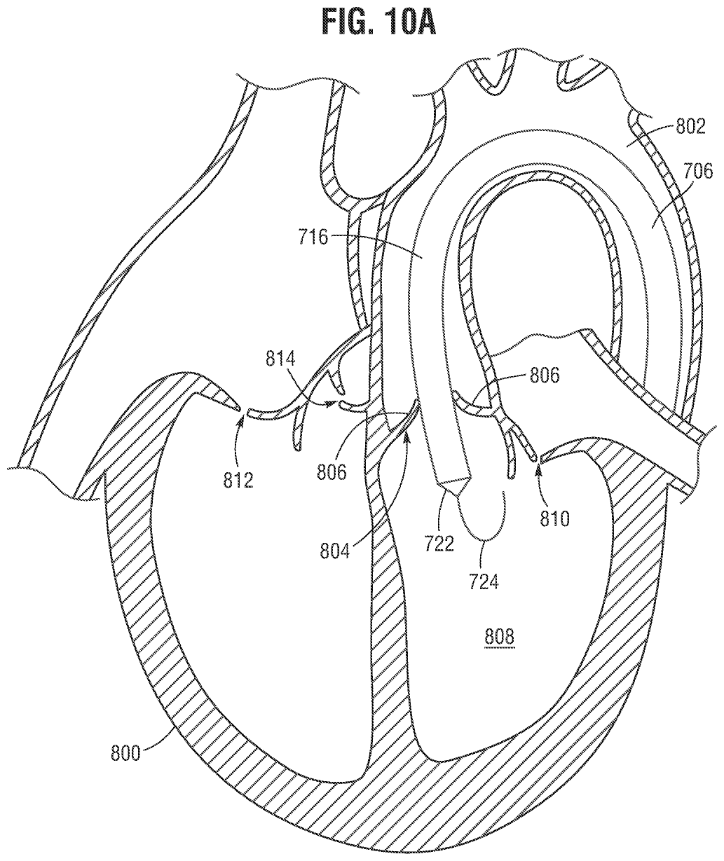

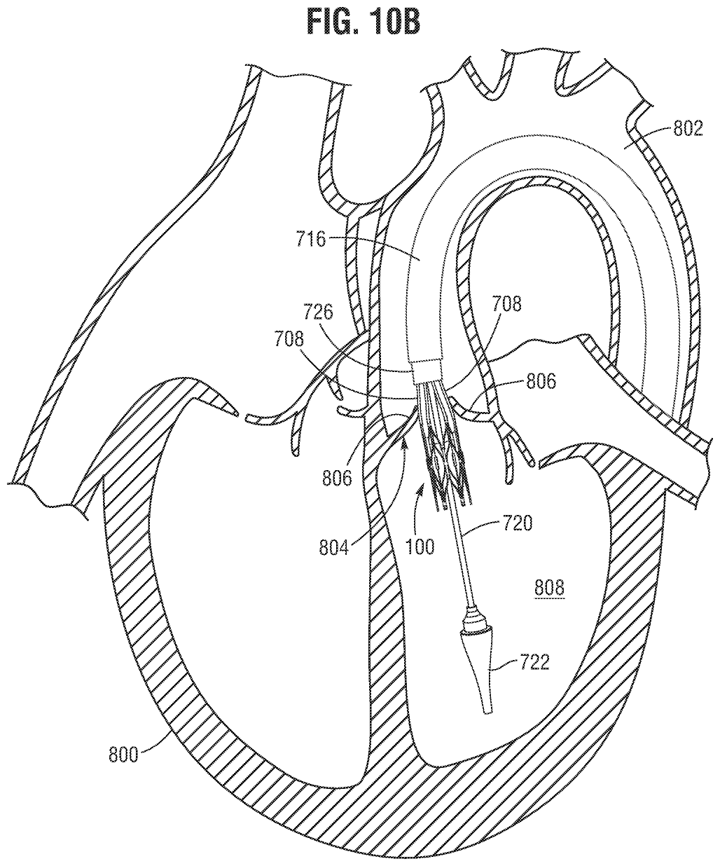

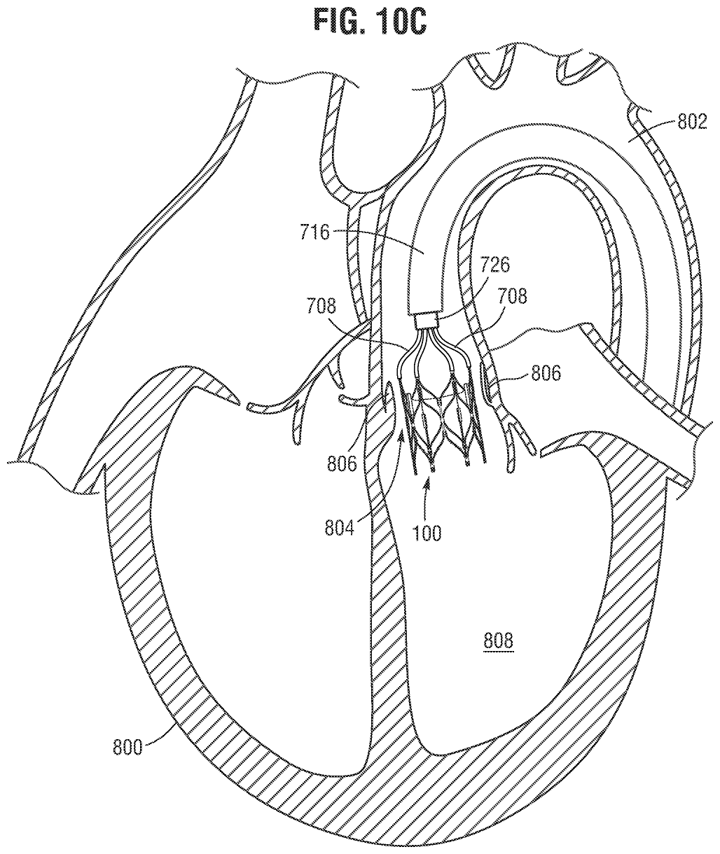

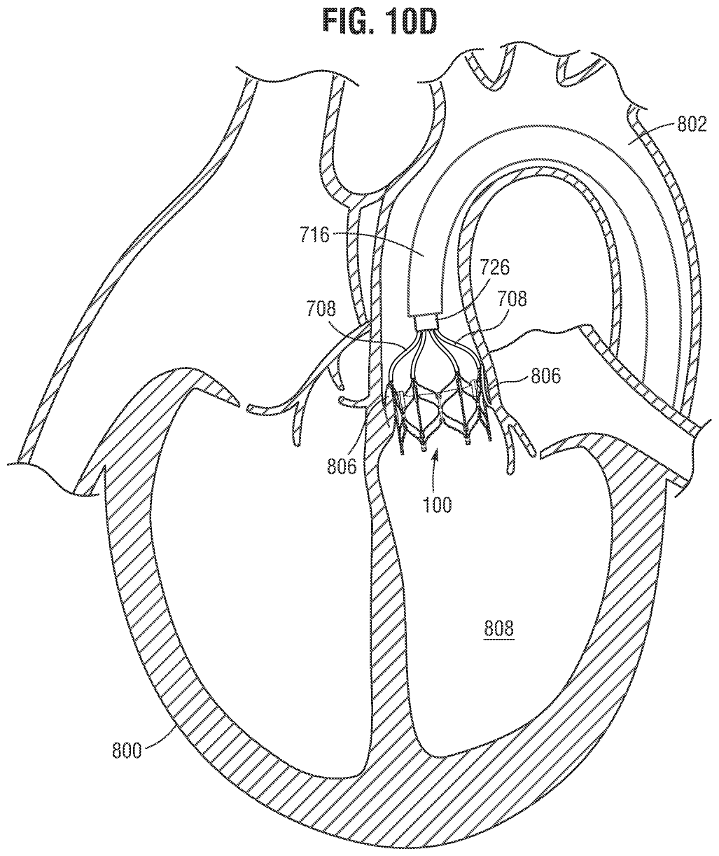

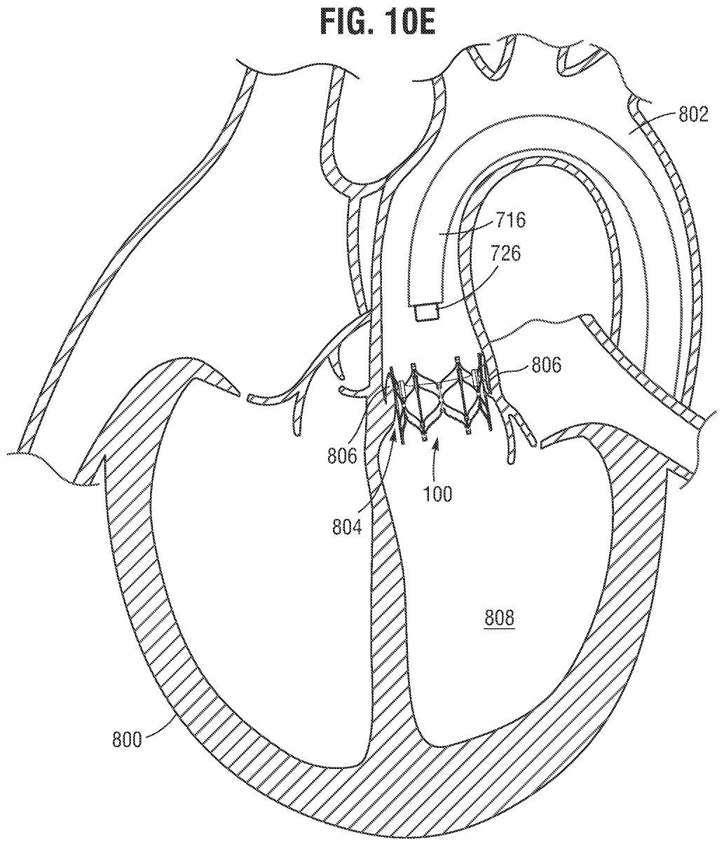

FIGS. 10A-10E illustrates exemplary implantation of a prosthetic heart valve, such as prosthetic heart valve 100 or any variation thereof described herein, using delivery apparatus 700. In FIG. 10A, the distal end portion 716 of the delivery apparatus 700 is inserted into the patient's vasculature such that the first shaft 706 extends through the ascending aorta 802 and such that the nosecone 722 extends through the existing valvular structure 804 (e.g., the annulus of the native aortic valve in FIG. 10A) and into the left ventricle 808 of the patient's heart 800. A guidewire 724 (e.g., extending through guidewire lumen 720) can initially be extended through the ascending aorta 802 and used to guide and position the distal end portion of the delivery apparatus 700 within a central region of the valvular structure 804 between leaflets 806 thereof. As shown in FIG. 10B, the prosthetic valve 100 can then be deployed from the distal end portion 716 of the delivery apparatus 700, for example, by moving the first shaft 706 proximally relative to a second shaft 726 and/or by moving the second shaft 726 distally relative to the first shaft 706. The first shaft 706 can be moved further proximally such that actuator assemblies 708 are exposed from the distal end portion 716.

The release of the prosthetic heart valve 100 from the distal end portion 716 of the delivery apparatus 700 can allow it to self-expand to its previously memorized configuration (e.g., shape-set diameter), as shown in FIG. 10C. As discussed above, the memorized configuration may be intermediate between the size of the valve 100 in its crimped state within the delivery apparatus 700 (e.g., having a diameter approaching 5 mm) and the size of the valve 100 in its implanted state within the valvular structure 804 (e.g., having a diameter approaching 30 mm). For example, the memorized configuration is partially-expanded state having a diameter of about 10 mm.

The delivery apparatus 700 can then be utilized to further expand the valve from the memorized configuration to the radially-expanded configuration, for example, by using actuator assemblies 708 coupled to the frame to apply axially forces urging the inflow and outflow ends of the valve 100 toward each other (e.g., as described above with respect to any of FIGS. 7A-8). As the valve 100 further expands, the female locking member 150 displaces into engagement with the male locking member to thereby retain the valve 100 in the radially-expanded configuration within the valvular structure 804. Once the prosthetic valve 100 has been fully expanded and secured, it can then be released from the delivery apparatus 700, as shown in FIG. 10E. The release can be accomplished by de-coupling the actuator assemblies 708 from the frame, after which the actuator assemblies and the second shaft 726 can be retracted into the distal end portion 716 of the first shaft, and the delivery apparatus 700 can be removed from the patient's body.

It should be appreciated that the distal end of the prosthetic valve 100 is positioned farther from the handle 704 of the delivery apparatus 700 than the proximal end of the prosthetic valve 100. That is, the distal end is configured to be positioned deeper within the patient's vasculature. In the illustrated examples, the distal end of the prosthetic valve 100 is thus the inflow end 108 of the prosthetic valve 100, and the proximal end of the prosthetic valve 100 is the outflow end 106 of the valve 100, such as when the prosthetic valve 100 is configured to replace a native aortic valve 804 and the prosthetic valve 100 is delivered to the native aortic valve via a retrograde, transfemoral delivery approach (e.g., through a femoral artery and the aorta). However, in other implementations, the distal end may be the outflow end 106 of the prosthetic valve 100 and the proximal end may be the inflow end 108 of the prosthetic valve 100, such as when the prosthetic valve 100 is delivered to the native aortic valve 804 via a transapical delivery approach, or when the prosthetic valve is configured to replace a native mitral valve 810 and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

Although the description above has focused on implantation of prosthetic heart valves at the aortic position (e.g., within native aortic valve 804), implementations of the disclosed subject matter are not limited thereto. Indeed, any of the prosthetic heart valve examples disclosed herein can be implanted at or within any of the native heart valves, including the aortic valve 804, the pulmonary valve 814, the mitral valve 810, and the tricuspid valve 812. Moreover, although the description above has focused on implantation in a native heart valve, any of the prosthetic heart valve examples disclosed herein can also be used in a Valve-in-Valve (ViV) procedure, where a new prosthetic heart valve is mounted within an existing (previously implanted), degrading prosthetic heart valve in order to restore proper function.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising a valvular structure comprising a plurality of leaflets; and a frame configured to support the valvular structure and to move between a radially-compressed configuration and a radially-expanded configuration, wherein the frame comprises a central longitudinal axis, a first axial end portion, a second axial end portion, a plurality of cells, and a plurality of struts, wherein the central longitudinal axis extends from the first axial end portion to the second axial end portion, wherein the plurality of cells are distributed circumferentially around the frame and are formed by the plurality of struts, wherein the plurality of struts includes a support arm, a first locking member, and a second locking member, wherein the support arm includes a fixed end portion extending from the first axial end portion and a free end portion disposed toward the second axial end portion relative to the fixed end portion and comprises the first locking member, wherein the fixed end portion has a first rotational position and the free end portion has a second rotational position which is rotationally offset relative to the first rotational position, wherein when the frame is in the radially-compressed configuration, the second locking member is disposed farther toward the second axial end portion and spaced apart from the first locking member, and wherein when the frame is in the radially-expanded configuration, the second locking member engages the first locking member, thereby restricting the frame from moving from the radially-expanded configuration to the radially-compressed configuration.

Example 2. The prosthetic heart valve of any example herein, particularly Example 1, wherein the second rotational position is rotationally offset relative to the first rotational position by less than 360 degrees.

Example 3. The prosthetic heart valve of any example herein, particularly any one of Examples 1-2, wherein the second rotational position is rotationally offset relative to the first rotational position by 90 degrees.

Example 4. The prosthetic heart valve of any example herein, particularly any one of Examples 1-2, wherein the second rotational position is rotationally offset relative to the first rotational position by 270 degrees.

Example 5. The prosthetic heart valve of any example herein, particularly Example 1, wherein the second rotational position is rotationally offset relative to the first rotational position by more than 360 degrees.

Example 6. The prosthetic heart valve of any example herein, particularly any one of Examples 1 or 5, wherein the second rotational position is rotationally offset relative to the first rotational position by 450 degrees.

Example 7. The prosthetic heart valve of any example herein, particularly any one of Examples 1 or 5, wherein the second rotational position is rotationally offset relative to the first rotational position by 630 degrees.

Example 8. The prosthetic heart valve of any example herein, particularly any one of Examples 1-7, wherein the first locking member comprises a first jaw and a second jaw spaced from each other by a gap, wherein the first jaw and the second jaw include one or more teeth protruding into the gap, and wherein the second locking member includes one or more openings configured to receive the one or more teeth of the first locking member when the frame is in the radially-expanded configuration.

Example 9. The prosthetic heart valve of any example herein, particularly any one of Examples 1-7, wherein the first locking member comprises a first jaw and a second jaw spaced from each other by a gap, wherein the first jaw and the second jaw include one or more teeth protruding into the gap, and wherein the second locking member includes one or more recesses configured to receive the one or more teeth of the first locking member when the frame is in the radially-expanded configuration.

Example 10. The prosthetic heart valve of any example herein, particularly any one of Examples 8-9, wherein the first jaw is disposed radially inwardly relative to the second jaw.

Example 11. The prosthetic heart valve of any example herein, particularly any one of Examples 1-10, wherein the plurality of struts are integrally formed as a unitary structure.

Example 12. The prosthetic heart valve of any example herein, particularly any one of Examples 1-11, further comprising a rotatable actuation member coupled to the frame, wherein rotating the rotatable actuation member in a first direction relative to the frame increases a diameter of the frame, and wherein rotating the rotatable actuation member in a second direction relative to the frame decreases the diameter of the frame.

Example 13. The prosthetic heart valve of any example herein, particularly any one of Examples 1-11, further comprising a lumen extending through the frame and configured to receiving an actuation member of a delivery apparatus that can be releasably coupled to the first axial end portion the frame of the prosthetic heart valve, wherein moving the actuation member of the delivery apparatus in a first axial direction relative to the second axial end portion of the frame increases a diameter of the frame, and wherein moving actuation member of the delivery apparatus in a second axial direction relative to the second axial end portion of the frame decreases the diameter of the frame.

Example 14. A prosthetic heart valve comprising: a frame having a central axis and comprising a plurality of first cells arranged along a circumferential direction of the frame; and a valvular structure supported within the frame and comprising a plurality of leaflets, wherein each first cell is formed by a plurality of interconnected first struts and has an inner cell disposed within an area bounded by the first struts of the first cell, each inner cell is formed by a plurality of interconnected second struts, each inner cell has a first locking member at an end of a support arm and a second locking member, the support arm extending axially from a first axial end of the respective inner cell toward a second axial end of the respective inner cell that is opposite the first axial end, the second locking member being at or adjacent to the second axial end of the respective inner cell, each first locking member has a twisted orientation such that a first part of the first locking member is disposed radially inward of the first axial end and a second part of the first locking member is disposed radially outward of the first axial end, and the frame is radially compressible and expandable between a radially-compressed configuration, where the first locking member is spaced from the second locking member along an axial direction of the frame, and a radially-expanded configuration, where the first and second locking members engage with each other to lock the frame in the radially-expanded configuration.

Example 15. The prosthetic heart valve of any example herein, particularly Example 14, wherein, for each inner cell: one of the first and second locking members comprises a female ratchet member having first and second jaws spaced from each other by a gap, at least one of the first and second jaws having one or more teeth protruding into said gap, and the other of the first and second locking members comprises a male ratchet member having at least one opening or recess constructed to receive therein the one or more teeth when the corresponding female ratchet member engages with the male ratchet member.

Example 16. The prosthetic heart valve of any example herein, particularly Example 15, wherein each male ratchet member has multiple openings or recesses serially arranged along the axial direction.

Example 17. The prosthetic heart valve of any example herein, particularly Example 16, wherein: each of the first and second jaws of the female ratchet member has multiple teeth serially arranged along the axial direction, and when the frame is in the radially-expanded configuration, each tooth of the female ratchet member is received by a respective one of the openings or recesses of the male ratchet member.

Example 18. The prosthetic heart valve of any example herein, particularly Example 17, wherein a thickness of the male ratchet member is greater than a distance between facing teeth of the female ratchet member.

Example 19. The prosthetic heart valve of any example herein, particularly any one of Examples 14-18, wherein, for each first cell: the first axial end of the inner cell is coupled to a first axial end of the corresponding first cell by a first vertical strut, the second axial end of the inner cell is coupled to a second axial end of the corresponding first cell by a second vertical strut, and intermediate portions of the inner cell between the first and second axial ends of the inner cell are coupled to adjacent intermediate portions of the corresponding first cell between the first and second axial ends of the first cell.

Example 20. The prosthetic heart valve of any example herein, particularly any one of Examples 14-18, wherein, for each first cell: the first axial end of the inner cell is coupled to a first axial end of the corresponding first cell by a first vertical strut, intermediate portions of the inner cell between the first and second axial ends of the inner cell are coupled to adjacent intermediate portions of the corresponding first cell between the first and second axial ends of the first cell, and an open region is disposed between the second axial end of the first cell and the second axial end of the inner cell, such that, in the radially-expanded configuration, at least a leading axial end of the first locking member is disposed in the open region.

Example 21. The prosthetic heart valve of any example herein, particularly any one of Examples 14-20, wherein, for each first cell, an actuator conduit extends through the first cell and corresponding inner cell from one axial end to an opposite axial end thereof.

Example 22. The prosthetic heart valve of any example herein, particularly any one of Examples 14-20, wherein: for each of a first subset of first cells, an actuator conduit extends through the first cell and corresponding inner cell from one axial end to an opposite axial end thereof, for each of a second subset of first cells, no actuator conduit extends through the first cell and corresponding inner cell, and the cells of the first and second subsets are alternately arranged along the circumferential direction.

Example 23. The prosthetic heart valve of any example herein, particularly any one of Examples 14-20, wherein: the frame further comprises a plurality of second cells alternately arranged with the first cells along the circumferential direction, each second cell lacking a first locking member and support arm, and for each of the first and second cells, an actuator conduit extends therethrough from one axial end to an opposite axial end thereof.

Example 24. The prosthetic heart valve of any example herein, particularly any one of Examples 14-20, wherein: the frame further comprises a plurality of second cells alternately arranged with the first cells along the circumferential direction, each second cell lacking a first locking member and support arm, for each of the second cells, an actuator conduit extends through the second cell from one axial end to an opposite axial end thereof, and for each of the first cells, no actuator conduit extends through the first cell and corresponding inner cell.

Example 25. The prosthetic heart valve of any example herein, particularly any one of Examples 14-20, wherein: the frame further comprises a plurality of second cells alternately arranged with the first cells along the circumferential direction, each second cell lacking a first locking member and support arm, for each of the first cells, an actuator conduit extends through the first cell and corresponding inner cell from one axial end to an opposite axial end thereof, and for each of the second cells, no actuator conduit extends through the second cell.

Example 26. The prosthetic heart valve of any example herein, particularly any one of Examples 23-25, wherein a shape and size of each second cell is substantially the same as that of each first cell.

Example 27. The prosthetic heart valve of any example herein, particularly any one of Examples 23-25, wherein a shape and size of each second cell is different than that of each first cell.

Example 28. The prosthetic heart valve of any example herein, particularly any one of Examples 21-27, wherein at least a portion of each actuator conduit is threaded so as to engage with a threaded portion of an actuation rod of a delivery apparatus in order to compress or expand the frame.

Example 29. The prosthetic heart valve of any example herein, particularly any one of Examples 14-28, wherein the frame is formed of a shape memory material.

Example 30. The prosthetic heart valve of any example herein, particularly Example 29, wherein the frame has been shape set to have a memorized configuration that is intermediate between the radially-compressed and radially-expanded configurations.

Example 31. The prosthetic heart valve of any example herein, particularly any one of Examples 14-30, wherein the frame is an annular frame.

Example 32. The prosthetic heart valve of any example herein, particularly any one of Examples 14-31, wherein the valvular structure is a bicuspid structure with two leaflets and two commissure tab assemblies, and the valvular structure is coupled to the frame via the commissure tab assemblies on diametrically opposite sides of the frame from each other.

Example 33. The prosthetic heart valve of any example herein, particularly any one of Examples 14-31, wherein the valvular structure is a tricuspid structure with three leaflets and three commissure tab assemblies, and the valvular structure is coupled to the frame via the three commissure tab assemblies equally spaced along the circumferential direction of the frame.

Example 34. The prosthetic heart valve of any example herein, particularly any one of Examples 14-33, wherein the prosthetic heart valve is constructed for implantation in a native heart valve or a previously-implanted prosthetic heart valve within a patient.

Example 35. The prosthetic heart valve of any example herein, particularly any one of Examples 14-34, wherein the prosthetic heart valve is constructed for implantation at an aortic position, a mitral position, a tricuspid position, or a pulmonary position.

Example 36. A prosthetic heart valve comprising: a frame having a central axis and comprising a plurality of first cells arranged along a circumferential direction of the frame; and a valvular structure supported within the frame and comprising a plurality of leaflets, wherein each first cell is formed by a plurality of interconnected first struts, each first cell has a first locking member at an end of a support arm and a second locking member, the support arm extending axially from a first axial end of the respective first cell toward a second axial end of the respective first cell that is opposite the first axial end, the second locking member being at or adjacent to the second axial end of the respective first cell, each first locking member has a twisted orientation such that a first part of the first locking member is disposed radially inward of the first axial end and a second part of the first locking member is disposed radially outward of the first axial end, and the frame is radially compressible and expandable between a radially-compressed configuration, where the first locking member is spaced from the second locking member along an axial direction of the frame, and a radially-expanded configuration, where the first and second locking members engage with each other to lock the frame in the radially-expanded configuration.

Example 37. The prosthetic heart valve of any example herein, particularly Example 36, wherein, for each first cell: one of the first and second locking members comprises a female ratchet member having first and second jaws spaced from each other by a gap, at least one of the first and second jaws having one or more teeth protruding into said gap, and the other of the first and second locking members comprises a male ratchet member having at least one opening or recess constructed to receive therein the one or more teeth when the corresponding female ratchet member engages with the male ratchet member.

Example 38. The prosthetic heart valve of any example herein, particularly Example 37, wherein each of the first and second jaws of the female ratchet member has multiple teeth serially arranged along the axial direction.

Example 39. The prosthetic heart valve of any example herein, particularly Example 38, wherein a thickness of the male ratchet member is greater than a distance between facing teeth of the female ratchet member.

Example 40. The prosthetic heart valve of any example herein, particularly any one of Examples 36-39, wherein, for each first cell, the male ratchet member is formed by a union or junction member interconnecting first struts at the second axial end of the first cell.

Example 41. The prosthetic heart valve of any example herein, particularly any one of Examples 36-40, wherein, for each first cell, an actuator conduit extends through the first cell from the second axial end to the first axial end.

Example 42. The prosthetic heart valve of any example herein, particularly any one of Examples 36-40, wherein: for each of a first subset of first cells, an actuator conduit extends through the first cell from the second axial end to the first axial end, for each of a second subset of first cells, no actuator conduit extends through the first cell, and the cells of the first and second subsets are alternately arranged along the circumferential direction.

Example 43. The prosthetic heart valve of any example herein, particularly any one of Examples 36-40, wherein: the frame further comprises a plurality of second cells alternately arranged with the first cells along the circumferential direction, each second cell lacking a first locking member and support arm, and for each of the first and second cells, an actuator conduit extends therethrough from one axial end to an opposite axial end.

Example 44. The prosthetic heart valve of any example herein, particularly any one of Examples 36-40, wherein: the frame further comprises a plurality of second cells alternately arranged with the first cells along the circumferential direction, each second cell lacking a first locking member and support arm, for each of the second cells, an actuator conduit extends through the second cell from one axial end to an opposite axial end thereof, and for each of the first cells, no actuator conduit extends through the first cell.

Example 45. The prosthetic heart valve of any example herein, particularly any one of Examples 36-40, wherein: the frame further comprises a plurality of second cells alternately arranged with the first cells along the circumferential direction, each second cell lacking a first locking member and support arm, for each of the first cells, an actuator conduit extends through the first cell from the first axial end to the second axial end, and for each of the second cells, no actuator conduit extends through the second cell.

Example 46. The prosthetic heart valve of any example herein, particularly any one of Examples 43-45, wherein a shape and size of each second cell is substantially the same as that of each first cell.

Example 47. The prosthetic heart valve of any example herein, particularly any one of Examples 43-45, wherein a shape and size of each second cell is different than that of each first cell.

Example 48. The prosthetic heart valve of any example herein, particularly any one of Examples 41-47, wherein at least a portion of each actuator conduit is threaded so as to engage with a threaded portion of an actuation rod of a delivery apparatus in order to compress or expand the frame.

Example 49. The prosthetic heart valve of any example herein, particularly any one of Examples 36-48, wherein the frame is formed of a shape memory material.

Example 50. The prosthetic heart valve of any example herein, particularly Example 49, wherein the frame has been shape set to have a memorized configuration that is intermediate between the radially-compressed and radially-expanded configurations.

Example 51. The prosthetic heart valve of any example herein, particularly any one of Examples 36-50, wherein the frame is an annular frame.

Example 52. The prosthetic heart valve of any example herein, particularly any one of Examples 36-51, wherein the valvular structure is a bicuspid structure with two leaflets and two commissure tab assemblies, and the valvular structure is coupled to the frame via the commissure tab assemblies on diametrically opposite sides of the frame from each other.

Example 53. The prosthetic heart valve of any example herein, particularly any one of Examples 36-51, wherein the valvular structure is a tricuspid structure with three leaflets and three commissure tab assemblies, and the valvular structure is coupled to the frame via the three commissure tab assemblies equally spaced along the circumferential direction of the frame.

Example 54. The prosthetic heart valve of any example herein, particularly any one of Examples 36-53, wherein the prosthetic heart valve is constructed for implantation in a native heart valve or a previously-implanted prosthetic heart valve within a patient.

Example 55. The prosthetic heart valve of any example herein, particularly any one of Examples 36-54, wherein the prosthetic heart valve is constructed for implantation at an aortic position, a mitral position, a tricuspid position, or a pulmonary position.

Example 56. A prosthetic heart valve comprising: a frame having a central axis and comprising a plurality of first cells arranged along a circumferential direction of the frame; and a valvular structure supported within the frame and comprising a plurality of leaflets, wherein each first cell is formed by a plurality of interconnected first struts, each first cell has a first locking member at an end of a first support arm and a second locking member at an end of a second support arm, the first support arm extending axially from a first axial end of the respective first cell toward a second axial end of the respective first cell that is opposite the first axial end, the second support arm extending axially from the second axial end of the respective first cell toward the first axial end of the respective first cell, each of the first and second locking members has a twisted orientation, and the frame is radially compressible and expandable between a radially-compressed configuration, where the first locking member is spaced from the second locking member along an axial direction of the frame, and a radially-expanded configuration, where the first and second locking members engage with each other to lock the frame in the radially-expanded configuration.

Example 57. The prosthetic heart valve of any example herein, particularly Example 56, wherein the twisted orientation of the first locking member or the second locking member is such that a first part thereof is disposed radially inward of the respective axial end and a second part thereof is disposed radially outward of the respective axial end.

Example 58. The prosthetic heart valve of any example herein, particularly any one of Examples 56-57, wherein, for each first cell: one of the first and second locking members comprises a female ratchet member having first and second jaws spaced from each other by a gap, at least one of the first and second jaws having one or more teeth protruding into said gap, and the other of the first and second locking members comprises a male ratchet member having at least one opening or recess constructed to receive therein the one or more teeth when the corresponding female ratchet member engages with the male ratchet member.

Example 59. The prosthetic heart valve of any example herein, particularly Example 58, wherein each of the first and second jaws of the female ratchet member has multiple teeth serially arranged along the axial direction.

Example 60. The prosthetic heart valve of any example herein, particularly any one of Examples 58-59, wherein the male ratchet member has multiple recesses serially arranged along the axial direction.

Example 61. The prosthetic heart valve of any example herein, particularly any one of Examples 56-60, wherein, for each first cell, an actuator conduit extends through the first cell from the second axial end to the first axial end.

Example 62. The prosthetic heart valve of any example herein, particularly any one of Examples 56-60, wherein: for each of a first subset of first cells, an actuator conduit extends through the first cell from the second axial end to the first axial end, for each of a second subset of first cells, no actuator conduit extends through the first cell, and the cells of the first and second subsets are alternately arranged along the circumferential direction.

Example 63. The prosthetic heart valve of any example herein, particularly any one of Examples 56-60, wherein: the frame further comprises a plurality of second cells alternately arranged with the first cells along the circumferential direction, each second cell lacking first and second locking members, and for each of the first and second cells, an actuator conduit extends therethrough from one axial end to an opposite axial end.

Example 64. The prosthetic heart valve of any example herein, particularly any one of Examples 56-60, wherein: the frame further comprises a plurality of second cells alternately arranged with the first cells along the circumferential direction, each second cell lacking a first locking member and support arm, for each of the second cells, an actuator conduit extends through the second cell from one axial end to an opposite axial end thereof, and for each of the first cells, no actuator conduit extends through the first cell.

Example 65. The prosthetic heart valve of any example herein, particularly any one of Examples 56-60, wherein: the frame further comprises a plurality of second cells alternately arranged with the first cells along the circumferential direction, each second cell lacking a first locking member and support arm, for each of the first cells, an actuator conduit extends through the first cell from the first axial end to the second axial end, and for each of the second cells, no actuator conduit extends through the second cell.

Example 66. The prosthetic heart valve of any example herein, particularly any one of Examples 63-65, wherein a shape and size of each second cell is substantially the same as that of each first cell.

Example 67. The prosthetic heart valve of any example herein, particularly any one of Examples 63-65, wherein a shape and size of each second cell is different than that of each first cell.

Example 68. The prosthetic heart valve of any example herein, particularly any one of Examples 61-67, wherein at least a portion of each actuator conduit is threaded so as to engage with a threaded portion of an actuation rod of a delivery apparatus in order to compress or expand the frame.

Example 69. The prosthetic heart valve of any example herein, particularly any one of Examples 56-68, wherein the frame is formed of a shape memory material.

Example 70. The prosthetic heart valve of any example herein, particularly Example 69, wherein the frame has been shape set to have a memorized configuration that is intermediate between the radially-compressed and radially-expanded configurations.

Example 71. The prosthetic heart valve of any example herein, particularly any one of Examples 56-70, wherein the frame is an annular frame.

Example 72. The prosthetic heart valve of any example herein, particularly any one of Examples 56-71, wherein the valvular structure is a bicuspid structure with two leaflets and two commissure tab assemblies, and the valvular structure is coupled to the frame via the commissure tab assemblies on diametrically opposite sides of the frame from each other.

Example 73. The prosthetic heart valve of any example herein, particularly any one of Examples 56-71, wherein the valvular structure is a tricuspid structure with three leaflets and three commissure tab assemblies, and the valvular structure is coupled to the frame via the three commissure tab assemblies equally spaced along the circumferential direction of the frame.

Example 74. The prosthetic heart valve of any example herein, particularly any one of Examples 56-73, wherein the prosthetic heart valve is constructed for implantation in a native heart valve or a previously-implanted prosthetic heart valve within a patient.

Example 75. The prosthetic heart valve of any example herein, particularly any one of Examples 56-74, wherein the prosthetic heart valve is constructed for implantation at an aortic position, a mitral position, a tricuspid position, or a pulmonary position.

Example 76. A prosthetic heart valve comprising: a frame having a central axis and comprising a plurality of first cells arranged along a circumferential direction of the frame; and a valvular structure supported within the frame and comprising a plurality of leaflets, wherein each first cell is formed by a plurality of interconnected first struts, the frame is radially compressible and expandable between a radially-compressed configuration and a radially-expanded configuration, and the frame comprises ratchet means for locking the frame in the radially-expanded configuration.

Example 77. The prosthetic heart valve of any example herein, particularly Example 76, wherein: each ratchet means comprises a female ratchet member and a male ratchet member, the female ratchet member has first and second jaws spaced from each other by a gap, at least one of the first and second jaws having one or more teeth protruding into said gap, and the male ratchet member has at least one opening or recess constructed to receive therein the one or more teeth when the corresponding female ratchet member engages with the male ratchet member.

Example 78. The prosthetic heart valve of any example herein, particularly any one of Examples 76-77, wherein, for each first cell, an actuator conduit extends through the first cell and corresponding inner cell from one axial end to an opposite axial end thereof.

Example 79. The prosthetic heart valve of any example herein, particularly any one of Examples 76-77, wherein: for each of a first subset of first cells, an actuator conduit extends through the first cell and corresponding inner cell from one axial end to an opposite axial end thereof, for each of a second subset of first cells, no actuator conduit extends through the first cell and corresponding inner cell, and the cells of the first and second subsets are alternately arranged along the circumferential direction.

Example 80. The prosthetic heart valve of any example herein, particularly any one of Examples 76-77, wherein: the frame further comprises a plurality of second cells alternately arranged with the first cells along the circumferential direction, each second cell lacking a ratchet means, and for each of the first and second cells, an actuator conduit extends therethrough from one axial end to an opposite axial end thereof.

Example 81. The prosthetic heart valve of any example herein, particularly any one of Examples 76-77, wherein: the frame further comprises a plurality of second cells alternately arranged with the first cells along the circumferential direction, each second cell lacking a ratchet means, for each of the second cells, an actuator conduit extends through the second cell from one axial end to an opposite axial end thereof, and for each of the first cells, no actuator conduit extends through the first cell and corresponding inner cell.

Example 82. The prosthetic heart valve of any example herein, particularly any one of Examples 76-77, wherein: the frame further comprises a plurality of second cells alternately arranged with the first cells along the circumferential direction, each second cell lacking a ratchet means, for each of the first cells, an actuator conduit extends through the first cell and corresponding inner cell from one axial end to an opposite axial end thereof, and for each of the second cells, no actuator conduit extends through the second cell.

Example 83. The prosthetic heart valve of any example herein, particularly any one of Examples 80-82, wherein a shape and size of each second cell is substantially the same as that of each first cell.

Example 84. The prosthetic heart valve of any example herein, particularly any one of Examples 80-82, wherein a shape and size of each second cell is different than that of each first cell.

Example 85. The prosthetic heart valve of any example herein, particularly any one of Examples 78-84, wherein at least a portion of each actuator conduit is threaded so as to engage with a threaded portion of an actuation rod of a delivery apparatus in order to compress or expand the frame.

Example 86. The prosthetic heart valve of any example herein, particularly any one of Examples 76-85, wherein the frame is formed of a shape memory material.

Example 87. The prosthetic heart valve of any example herein, particularly Example 86, wherein the frame has been shape set to have a memorized configuration that is intermediate between the radially-compressed and radially-expanded configurations.

Example 88. The prosthetic heart valve of any example herein, particularly any one of Examples 76-87, wherein the frame is an annular frame.

Example 89. The prosthetic heart valve of any example herein, particularly any one of Examples 76-88, wherein the valvular structure is a bicuspid structure with two leaflets and two commissure tab assemblies, and the valvular structure is coupled to the frame via the commissure tab assemblies on diametrically opposite sides of the frame from each other.

Example 90. The prosthetic heart valve of any example herein, particularly any one of Examples 76-88, wherein the valvular structure is a tricuspid structure with three leaflets and three commissure tab assemblies, and the valvular structure is coupled to the frame via the three commissure tab assemblies equally spaced along the circumferential direction of the frame.

Example 91. The prosthetic heart valve of any example herein, particularly any one of Examples 76-90, wherein the prosthetic heart valve is constructed for implantation in a native heart valve or a previously-implanted prosthetic heart valve within a patient.

Example 92. The prosthetic heart valve of any example herein, particularly any one of Examples 76-91, wherein the prosthetic heart valve is constructed for implantation at an aortic position, a mitral position, a tricuspid position, or a pulmonary position.

Example 93. An assembly comprising: a delivery apparatus comprising an elongated shaft; and the prosthetic heart valve of any one of Examples 1-92 releasably supported within the delivery apparatus in the radially-compressed configuration for delivery into a patient's body.

Example 94. The assembly of any example herein, particularly Example 93, wherein the delivery apparatus comprises a plurality of actuators, at least a portion of each actuator being disposed within a corresponding actuator conduit of the frame of the prosthetic heart valve, each actuator being configured to expand the frame to the radially-expanded configuration.

Example 95. The assembly of any example herein, particularly Example 94, wherein each actuator comprises a threaded rod.

Example 96. The assembly of any example herein, particularly Example 94, wherein each actuator comprises a suture or wire.

Example 97. A method of implanting a prosthetic heart valve in a patient's body, the method comprising: inserting a distal end of a delivery apparatus into vasculature of a patient, the delivery apparatus comprising an elongated shaft, the prosthetic heart valve of any example herein, particularly any one of Examples 1-92, the prosthetic heart valve being releasably supported within the delivery apparatus in the radially-compressed configuration; advancing the prosthetic heart valve to a desired implantation site; and using the delivery apparatus to expand the prosthetic heart valve to the radially-expanded configuration, thereby implanting the prosthetic heart valve at the desired implantation site.

Example 98. A method of implanting a prosthetic heart valve in a patient's body, the method comprising: inserting a distal end of a delivery apparatus into vasculature of a patient, the delivery apparatus comprising an elongated shaft, the prosthetic heart valve of any example herein, particularly any one of Examples 1-92, the prosthetic heart valve being releasably supported within the delivery apparatus in the radially-compressed configuration; advancing the prosthetic heart valve to a desired implantation site; deploying the prosthetic heart valve from the delivery apparatus such that the prosthetic heart valve self-expands to a previously shape-set configuration that is intermediate between the radially-compressed and radially expanded configurations; and using the delivery apparatus to further expand the prosthetic heart valve to the radially-expanded configuration, thereby implanting the prosthetic heart valve at the desired implantation site.

Example 99. The method of any example herein, particularly any one of Examples 97-98, wherein the delivery apparatus comprises a plurality of actuators, at least a portion of each actuator being disposed within a corresponding actuator conduit of the frame of the prosthetic heart valve, and the actuators are used to expand the frame to the radially-expanded configuration.

Example 100. The method of any example herein, particularly Example 99, wherein each actuator comprises a threaded rod.

Example 101. The method of any example herein, particularly Example 99, wherein each actuator comprises a suture or wire.

Example 102. The method of any example herein, particularly any one of Examples 97-101, wherein the advancing to the desired implantation site employs transfemoral, transventricular, transapical, or transseptal approaches.

Example 103. The method of any example herein, particularly any one of Examples 97-102, wherein the desired implantation site is a native heart valve or a previously-implanted prosthetic heart valve within the patient.

Example 104. The method of any example herein, particularly any one of Examples 97-103, wherein the desired implantation site is at an aortic position, a mitral position, a tricuspid position, or a pulmonary position within the patient.

Example 105. A method of fabricating a prosthetic heart valve, the method comprising: forming a frame having a plurality of first cells, each first cell comprising a plurality of interconnected first struts and an inner cell disposed within an area bounded by the first struts of the first cell, each inner cell comprising a plurality of interconnected second struts, a first locking member at an end of a support arm and a second locking member, the support arm extending from a first end of the respective inner cell toward a second end of the respective inner cell that is opposite the first end, the second locking member being at or adjacent to the second end of the respective inner cell, the frame being formed of a shape memory material, each first locking member being in an initial orientation; and shape-setting each first locking member to have a twisted orientation with respect to the corresponding inner cell.

Example 106. The method of any example herein, particularly Example 105, wherein the shape-setting comprises: at a temperature in excess of a transition temperature of the shape memory material, rotating each first locking member about a longitudinal axis of the support arm, such that the first locking member has the twisted orientation, with a first part of the first locking member disposed on one side of a plane of the respective first cell and a second part of the first locking member disposed on an opposite of the plane of the respective first cell; and cooling the frame to a temperature below the transition temperature to set each first locking member in the twisted orientation.

Example 107. The method of any example herein, particularly Example 106, wherein each first locking member is rotated 90° about the longitudinal axis of the corresponding support arm to provide the first locking member with the twisted orientation.

Example 108. The method of any example herein, particularly Example 105, wherein the shape-setting comprises: heating one or more portions of the frame to a temperature in excess of a transition temperature of the shape memory material; at the temperature in excess of the transition temperature, rotating each first locking member about a longitudinal axis of the respective support arm by an incremental amount; cooling the frame to a temperature below the transition temperature; and repeating the heating, the rotating, and the cooling one or more times until each first locking member is provided with the twisted orientation.

Example 109. The method of any example herein, particularly any one of Examples 105-108, wherein the frame is formed as flat structure with a linear array of first cells, and the method further comprises joining first cells at opposite ends of the linear array to form an annular structure.

Example 110. The method of any example herein, particularly any one of Examples 105-108, wherein the frame is formed as an annular structure with the first cells arranged along a circumferential direction of the frame.

Example 111. The method of any example herein, particularly any one of Examples 105-110, wherein the frame is formed to be radially compressible and expandable between a radially-compressed configuration and a radially-expanded configuration.

Example 112. The method of any example herein, particularly Example 111, wherein: in the radially-compressed configuration, the first locking member is spaced from the second locking member along an axial direction of the frame, and in the radially-expanded configuration, the first and second locking members engage with each other to lock the frame.

Example 113. The method of any example herein, particularly any one of Examples 105-112, further comprising: shape-setting the frame to have a memorized configuration that is intermediate between the radially-compressed and radially-expanded configurations.

Example 114. The method of any example herein, particularly Example 113, wherein the shape-setting the frame is performed at a same time as the shape-setting each first locking member.

Example 115. The method of any example herein, particularly any one of Examples 105-114, wherein: prior to the shape-setting, each first locking member has an orientation parallel to a plane of the respective first cell, and after the shape-setting, each first locking member has an orientation substantially perpendicular to the plane of the respective first cell.

Example 116. The method of any example herein, particularly any one of Examples 105-115, wherein the forming comprises cutting a solid starting material to form the first struts, the second struts, the first locking members, the support arms, and the second locking members.

Example 117. The method of any example herein, particularly Example 116, wherein the solid starting material is a flat panel or a tube.

Example 118. The method of any example herein, particularly any one of Examples 105-117, wherein the forming the frame is such that, for each inner cell: one of the first and second locking members comprises a female ratchet member having first and second jaws spaced from each other by a gap, at least one of the first and second jaws having one or more teeth protruding into said gap, and the other of the first and second locking members comprises a male ratchet member having at least one opening or recess constructed to receive therein the one or more teeth when the corresponding female ratchet member engages with the male ratchet member.

Example 119. The method of any example herein, particularly Example 118, wherein: the forming the frame is such that each male ratchet member has multiple openings or recesses serially arranged, and each of the first and second jaws of the female ratchet member has multiple teeth serially arranged, and when the frame is in the radially-expanded configuration, each tooth of the female ratchet member is received by a respective one of the openings or recesses of the male ratchet member.

Example 120. The method of any example herein, particularly any one of Examples 105-119, wherein the forming the frame is such that, for each first cell: the first end of the inner cell is coupled to a first end of the corresponding first cell by a first vertical strut, the second end of the inner cell is coupled to a second end of the corresponding first cell by a second vertical strut, and intermediate portions of the inner cell between the first and second ends of the inner cell are coupled to adjacent intermediate portions of the corresponding first cell between the first and second ends of the first cell.

Example 121. The method of any example herein, particularly any one of Examples 105-119, wherein the forming is such that, for each first cell: the first end of the inner cell is coupled to a first end of the corresponding first cell by a first vertical strut, intermediate portions of the inner cell between the first and second ends of the inner cell are coupled to adjacent intermediate portions of the corresponding first cell between the first and second ends of the first cell, and an open region is disposed between the second end of the first cell and the second end of the inner cell, such that, in the radially-expanded configuration, at least a leading end of the first locking member is disposed in the open region.

Example 122. The method of any example herein, particularly any one of Examples 105-121, further comprising, before or after the shape-setting each first locking member, forming an actuator conduit for each first cell, the actuator conduit extending through the first cell and corresponding inner cell from one end to an opposite end thereof.

Example 123. The method of any example herein, particularly any one of Examples 105-121, further comprising, before or after the shape-setting each first locking member: forming an actuator conduit for each of a first subset of the first cells without forming an actuator conduit in each of a second subset of the first cells, wherein each actuator conduit extends through the respective first cell and corresponding inner cell from one end to an opposite end thereof, and the first cells of the first and second subsets are alternately arranged in the frame.

Example 124. The method of any example herein, particularly any one of Examples 105-121, wherein: the forming the frame is such that the frame has a plurality of second cells alternately arranged with the first cells, each second cell lacking a first locking member and support arm; and the method further comprises forming an actuator conduit for each of the first and second cells, each actuator conduit extending through the respective first or second cell from one end thereof to an opposite end thereof.

Example 125. The method of any example herein, particularly any one of Examples 105-121, wherein: the forming the frame is such that the frame has a plurality of second cells alternately arranged with the first cells, each second cell lacking a first locking member and support arm; and the method further comprises forming an actuator conduit for each of the second cells without forming an actuator conduit in each of the first cells, each actuator conduit extending through the respective second cell from one end thereof to an opposite end thereof.

Example 126. The method of any example herein, particularly any one of Examples 105-121, wherein: the forming the frame is such that the frame has a plurality of second cells alternately arranged with the first cells, each second cell lacking a first locking member and support arm; and the method further comprises forming an actuator conduit for each of the first cells without forming an actuator conduit in each of the second cells, each actuator conduit extending through the respective first cell from one end thereof to an opposite end thereof.

Example 127. The method of any example herein, particularly any one of Examples 124-126, further comprising: shape-setting each first cell to have a first memorized configuration; and shape-setting each second cell to have a second memorized configuration, wherein a size of the first memorized configuration is different than a size of the second memorized configuration.

Example 128. The method of any example herein, particularly any one of Examples 122-127, wherein the forming the actuator conduit is such that at least a portion of the actuator conduit is threaded.

Example 129. The method of any example herein, particularly any one of Examples 105-128, further comprising: coupling a valvular structure to the frame, the valvular structure comprising a plurality of leaflets.

Example 130. The method of any example herein, particularly Example 129, wherein the valvular structure is a tricuspid structure with three leaflets and three commissure tab assemblies, and the valvular structure is coupled to the frame via the three commissure tab assemblies equally spaced along a circumferential direction of the frame.

Example 131. The method of any example herein, particularly Example 129, wherein the valvular structure is a bicuspid structure with two leaflets and two commissure tab assemblies, and the valvular structure is coupled to the frame via the commissure tab assemblies on diametrically opposite sides of the frame from each other.

Example 132. The method of any example herein, particularly any one of Examples 105-131, further comprising: attaching an inner skirt to an inner circumferential surface of the frame; attaching an outer skirt to an outer circumferential surface of the frame; or any combination of the above.

Example 133. A method of fabricating a prosthetic heart valve, the method comprising: forming a frame having a plurality of first cells and a plurality of first locking members, each first cell comprising a plurality of interconnected first struts and a second locking member at or adjacent to the second end of the respective first cell, each first locking member being at an end of a support arm that extends from a first end of a corresponding first cell, the frame being formed of a shape memory material, each first locking member being in an initial orientation; and shape-setting each first locking member to have a twisted orientation with respect to the corresponding first cell.

Example 134. The method of any example herein, particularly Example 133, wherein the shape-setting comprises: at a temperature in excess of a transition temperature of the shape memory material, rotating each first locking member about a longitudinal axis of the support arm, such that the first locking member has the twisted orientation, with a first part of the first locking member disposed on one side of a plane of the respective first cell and a second part of the first locking member disposed on an opposite of the plane of the respective first cell; and cooling the frame to a temperature below the transition temperature to set each first locking member in the twisted orientation.

Example 135. The method of any example herein, particularly Example 134, wherein each first locking member is rotated 90° about the longitudinal axis of the corresponding support arm to provide the first locking member with the twisted orientation.

Example 136. The method of any example herein, particularly Example 133, wherein the shape-setting comprises: heating one or more portions of the frame to a temperature in excess of a transition temperature of the shape memory material; at the temperature in excess of the transition temperature, rotating each first locking member about a longitudinal axis of the respective support arm by an incremental amount; cooling the frame to a temperature below the transition temperature; and repeating the heating, the rotating, and the cooling one or more times until each first locking member is provided with the twisted orientation.

Example 137. The method of any example herein, particularly any one of Examples 133-136, wherein: after the forming the frame, each first locking member is outside a boundary formed by the interconnected first struts of the corresponding first cell such that the support arm extends away from the second end of the first cell; and the method further comprises, prior to or after the shape-setting each first locking member, rotating each support arm about the first end of the corresponding first cell such that at least part of the first locking member is within the boundary formed by the interconnected first struts and the support arm extends toward the second end of the corresponding first cell.

Example 138. The method of any example herein, particularly any one of Examples 133-137, wherein the frame is formed as flat structure with a linear array of first cells, and the method further comprises joining first cells at opposite ends of the linear array to form an annular structure.

Example 139. The method of any example herein, particularly any one of Examples 133-137, wherein the frame is formed as an annular structure with the first cells arranged along a circumferential direction of the frame.

Example 140. The method of any example herein, particularly any one of Examples 133-139, wherein the frame is formed to be radially compressible and expandable between a radially-compressed configuration and a radially-expanded configuration.

Example 141. The method of any example herein, particularly Example 140, wherein: in the radially-compressed configuration, the first locking member is spaced from the second locking member along an axial direction of the frame, and in the radially-expanded configuration, the first and second locking members engage with each other to lock the frame.

Example 142. The method of any example herein, particularly any one of Examples 133-141, further comprising: shape-setting the frame to have a memorized configuration that is intermediate between the radially-compressed and radially-expanded configurations.

Example 143. The method of any example herein, particularly Example 142, wherein the shape-setting the frame is performed at a same time as the shape-setting each first locking member.

Example 144. The method of any example herein, particularly any one of Examples 133-143, wherein: prior to the shape-setting, each first locking member has an orientation parallel to a plane of the respective first cell, and after the shape-setting, each first locking member has an orientation substantially perpendicular to the plane of the respective first cell.

Example 145. The method of any example herein, particularly any one of Examples 133-144, wherein the forming comprises cutting a solid starting material to form the struts of the first cells, the first locking members, the support arms, and the second locking members.

Example 146. The method of any example herein, particularly Example 145, wherein the solid starting material is a flat panel or a tube.

Example 147. The method of any example herein, particularly any one of Examples 133-146, wherein the forming the frame is such that, for each pair of the first and second locking members: one of the pair comprises a female ratchet member having first and second jaws spaced from each other by a gap, at least one of the first and second jaws having one or more teeth protruding into said gap, and the other of the pair comprises a male ratchet member having at least one opening or recess constructed to receive therein the one or more teeth when the corresponding female ratchet member engages with the male ratchet member.

Example 148. The method of any example herein, particularly Example 147, wherein the forming the frame is such that each of the first and second jaws of the female ratchet member has multiple teeth serially arranged.

Example 149. The method of any example herein, particularly any one of Examples 147-148, wherein the forming the frame is such that, for each first cell, the male ratchet member is formed by a union or junction member interconnecting first struts at the second end of the first cell.

Example 150. The method of any example herein, particularly any one of Examples 133-149, further comprising, before or after the shape-setting each first locking member, forming an actuator conduit for each first cell and each first locking member, the actuator conduit extending through the first cell and the first locking member from one end to an opposite end thereof.

Example 151. The method of any example herein, particularly any one of Examples 133-149, further comprising, before or after the shape-setting each first locking member: forming an actuator conduit for each of a first subset of the first cells and first locking members without forming an actuator conduit in each of a second subset of the first cells and first locking members, wherein each actuator conduit extends through the respective first cell and corresponding first locking member from one end to an opposite end thereof, and the first cells of the first and second subsets are alternately arranged in the frame.

Example 152. The method of any example herein, particularly any one of Examples 133-149, wherein: the forming the frame is such that the frame has a plurality of second cells alternately arranged with the first cells, each second cell lacking a corresponding first locking member and support arm; and the method further comprises forming an actuator conduit for each of the first and second cells, each actuator conduit extending through the respective first or second cell from one end thereof to an opposite end thereof.

Example 153. The method of any example herein, particularly any one of Examples 133-149, wherein: the forming the frame is such that the frame has a plurality of second cells alternately arranged with the first cells, each second cell lacking a corresponding first locking member and support arm; and the method further comprises forming an actuator conduit for each of the second cells without forming an actuator conduit in each of the first cells and corresponding first locking members, each actuator conduit extending through the respective second cell from one end thereof to an opposite end thereof.

Example 154. The method of any example herein, particularly any one of Examples 133-149, wherein: the forming the frame is such that the frame has a plurality of second cells alternately arranged with the first cells, each second cell lacking a corresponding first locking member and support arm; and the method further comprises forming an actuator conduit for each of the first cells and corresponding first locking members without forming an actuator conduit in each of the second cells, each actuator conduit extending through the respective first cell and corresponding first locking member from one end thereof to an opposite end thereof.

Example 155. The method of any example herein, particularly any one of Examples 152-154, further comprising: shape-setting each first cell to have a first memorized configuration; and shape-setting each second cell to have a second memorized configuration, wherein a size of the first memorized configuration is different than a size of the second memorized configuration.

Example 156. The method of any example herein, particularly any one of Examples 150-155, wherein the forming the actuator conduit is such that at least a portion of the actuator conduit is threaded.

Example 157. The method of any example herein, particularly any one of Examples 133-156, further comprising: coupling a valvular structure to the frame, the valvular structure comprising a plurality of leaflets.

Example 158. The method of any example herein, particularly Example 157, wherein the valvular structure is a tricuspid structure with three leaflets and three commissure tab assemblies, and the valvular structure is coupled to the frame via the three commissure tab assemblies equally spaced along a circumferential direction of the frame.

Example 159. The method of any example herein, particularly Example 157, wherein the valvular structure is a bicuspid structure with two leaflets and two commissure tab assemblies, and the valvular structure is coupled to the frame via the commissure tab assemblies on diametrically opposite sides of the frame from each other.

Example 160. The method of any example herein, particularly any one of Examples 133-159, further comprising: attaching an inner skirt to an inner circumferential surface of the frame; attaching an outer skirt to an outer circumferential surface of the frame; or any combination of the above.

Example 161. A method of fabricating a prosthetic heart valve, the method comprising: forming a frame having a plurality of first cells, each first cell comprising a plurality of interconnected first struts, a first locking member at an end of a first support arm, and a second locking member at an end of a second support arm, the first support arm extending from a first end of the respective first cell toward a second end of the respective first cell that is opposite the first end, the second support arm extending axially from the second end of the respective first cell toward the first end of the respective first cell, the frame being formed of a shape memory material, each of the first and second locking members being in an initial orientation; and shape-setting each of the first and second locking members to have a twisted orientation with respect to the corresponding first cell.

Example 162. The method of any example herein, particularly Example 161, wherein the shape-setting comprises: at a temperature in excess of a transition temperature of the shape memory material, rotating each first locking member about a longitudinal axis of the first support arm, such that the first locking member has the twisted orientation, with a first part of the first locking member disposed on one side of a plane of the respective first cell and a second part of the first locking member disposed on an opposite of the plane of the respective first cell; at a temperature in excess of the transition temperature, rotating each second locking member about a longitudinal axis of the second support arm, such that the second locking member has the twisted orientation, with a first part of the second locking member disposed on one side of a plane of the respective first cell and a second part of the second locking member disposed on an opposite of the plane of the respective first cell; and cooling the frame to a temperature below the transition temperature to set each of the first and second locking members in the twisted orientation.

Example 163. The method of any example herein, particularly Example 162, wherein: each first locking member is rotated 90° about the longitudinal axis of the corresponding first support arm to provide the first locking member with the twisted orientation; and each second locking member is rotated 90° about the longitudinal axis of the corresponding second support arm to provide the second locking member with the twisted orientation.

Example 164. The method of any example herein, particularly Example 161, wherein the shape-setting comprises: heating one or more portions of the frame to a temperature in excess of a transition temperature of the shape memory material; at the temperature in excess of the transition temperature, rotating each first locking member and each second locking member about a longitudinal axis of the respective support arm by an incremental amount; cooling the frame to a temperature below the transition temperature; and repeating the heating, the rotating, and the cooling one or more times until each of the first and second locking members is provided with the twisted orientation.

Example 165. The method of any example herein, particularly any one of Examples 161-164, wherein the frame is formed as flat structure with a linear array of first cells, and the method further comprises joining first cells at opposite ends of the linear array to form an annular structure.

Example 166. The method of any example herein, particularly any one of Examples 161-164, wherein the frame is formed as an annular structure with the first cells arranged along a circumferential direction of the frame.

Example 167. The method of any example herein, particularly any one of Examples 161-164, wherein the frame is formed to be radially compressible and expandable between a radially-compressed configuration and a radially-expanded configuration.

Example 168. The method of any example herein, particularly Example 167, wherein: in the radially-compressed configuration, the first locking member is spaced from the second locking member along an axial direction of the frame, and in the radially-expanded configuration, the first and second locking members engage with each other to lock the frame.

Example 169. The method of any example herein, particularly any one of Examples 161-168, further comprising: shape-setting the frame to have a memorized configuration that is intermediate between the radially-compressed and radially-expanded configurations.

Example 170. The method of any example herein, particularly Example 169, wherein the shape-setting the frame is performed at a same time as the shape-setting each of the first and second locking members.

Example 171. The method of any example herein, particularly any one of Examples 161-170, wherein: prior to the shape-setting, each of the first and second locking members has an orientation parallel to a plane of the respective first cell, and after the shape-setting, each of the first and second locking members has an orientation substantially perpendicular to the plane of the respective first cell.

Example 172. The method of any example herein, particularly any one of Examples 161-171, wherein the forming comprises cutting a solid starting material to form the struts of the first cells, the first locking members, the first support arms, the second locking members, and the second support arms.

Example 173. The method of any example herein, particularly Example 172, wherein the solid starting material is a flat panel or a tube.

Example 174. The method of any example herein, particularly any one of Examples 161-173, wherein the forming the frame is such that, for each first cell: one of the first and second locking members comprises a female ratchet member having first and second jaws spaced from each other by a gap, at least one of the first and second jaws having one or more teeth protruding into said gap, and the other of the first and second locking members comprises a male ratchet member having at least one opening or recess constructed to receive therein the one or more teeth when the corresponding female ratchet member engages with the male ratchet member.

Example 175. The method of any example herein, particularly Example 174, wherein the forming the frame is such that: each of the first and second jaws of the female ratchet member has multiple teeth serially arranged; and the male ratchet member has multiple recesses serially arranged.

Example 176. The method of any example herein, particularly any one of Examples 161-175, further comprising, before or after the shape-setting each of the first and second locking members, forming an actuator conduit for each first cell, the actuator conduit extending through the first cell from one end to an opposite end thereof.

Example 177. The method of any example herein, particularly any one of Examples 161-175, further comprising, before or after the shape-setting each of the first and second locking members: forming an actuator conduit for each of a first subset of the first cells without forming an actuator conduit in each of a second subset of the first cells, wherein each actuator conduit extends through the respective first cell from one end to an opposite end thereof, and the first cells of the first and second subsets are alternately arranged in the frame.

Example 178. The method of any example herein, particularly any one of Examples 161-175, wherein: the forming the frame is such that the frame has a plurality of second cells alternately arranged with the first cells, each second cell lacking first and second locking members; and the method further comprises forming an actuator conduit for each of the first and second cells, each actuator conduit extending through the respective first or second cell from one end thereof to an opposite end thereof.

Example 179. The method of any example herein, particularly any one of Examples 161-175, wherein: the forming the frame is such that the frame has a plurality of second cells alternately arranged with the first cells, each second cell lacking first and second locking members; and the method further comprises forming an actuator conduit for each of the second cells without forming an actuator conduit in each of the first cells, each actuator conduit extending through the respective second cell from one end thereof to an opposite end thereof.

Example 180. The method of any example herein, particularly any one of Examples 161-175, wherein: the forming the frame is such that the frame has a plurality of second cells alternately arranged with the first cells, each second cell lacking first and second locking members; and the method further comprises forming an actuator conduit for each of the first cells without forming an actuator conduit in each of the second cells, each actuator conduit extending through the respective first cell from one end thereof to an opposite end thereof.

Example 181. The method of any example herein, particularly any one of Examples 178-180, further comprising: shape-setting each first cell to have a first memorized configuration; and shape-setting each second cell to have a second memorized configuration, wherein a size of the first memorized configuration is different than a size of the second memorized configuration.

Example 182. The method of any example herein, particularly any one of Examples 176-181, wherein the forming the actuator conduit is such that at least a portion of the actuator conduit is threaded.

Example 183. The method of any example herein, particularly any one of Examples 161-182, further comprising: coupling a valvular structure to the frame, the valvular structure comprising a plurality of leaflets.

Example 184. The method of any example herein, particularly Example 183, wherein the valvular structure is a tricuspid structure with three leaflets and three commissure tab assemblies, and the valvular structure is coupled to the frame via the three commissure tab assemblies equally spaced along a circumferential direction of the frame.

Example 185. The method of any example herein, particularly Example 183, wherein the valvular structure is a bicuspid structure with two leaflets and two commissure tab assemblies, and the valvular structure is coupled to the frame via the commissure tab assemblies on diametrically opposite sides of the frame from each other.

Example 186. The method of any example herein, particularly any one of Examples 161-185, further comprising: attaching an inner skirt to an inner circumferential surface of the frame; attaching an outer skirt to an outer circumferential surface of the frame; or any combination of the above.

Example 187. A frame for a prosthetic heart valve, comprising: a first axial end portion; a second axial end portion; and a plurality of struts extending from the first axial end portion to the second axial end portion and including a support arm, a first locking member, and a second locking member, wherein: the support arm includes a fixed end portion and a free end portion, the fixed end portion of the support arm is disposed at the first axial end portion of the frame and has a first rotational position, the free end portion is disposed toward the second axial end portion relative to the fixed end portion and has a second rotational position rotationally offset relative to the first rotational position of the fixed end portion, the first locking member extends from the free end portion of the support arm, the second locking member is disposed at the second axial end portion of the frame, the frame is radially expandable from a first diameter to a second diameter which is larger than the first diameter, when the frame is at the first diameter, the second locking member is disposed farther toward the second axial end portion and axially spaced apart from the first locking member, and when the frame is at the second diameter, the second locking member engages the first locking member such that the frame is prevented from moving from the second diameter to the first diameter.

Example 188. A stent comprising: a first axial end portion; a second axial end portion; and a plurality of struts extending from the first axial end portion to the second axial end portion and including a support arm, a first locking member, and a second locking member, wherein: the support arm includes a fixed end portion and a free end portion, the fixed end portion of the support arm is disposed at the first axial end portion and has a first rotational position, the free end portion is disposed toward the second axial end portion relative to the fixed end portion and has a second rotational position rotationally offset relative to the first rotational position of the fixed end portion, the first locking member extends from the free end portion of the support arm, the second locking member is disposed at the second axial end portion, the stent is radially expandable from a first diameter to a second diameter which is larger than the first diameter, when the stent is at the first diameter, the second locking member is disposed farther toward the second axial end portion and axially spaced apart from the first locking member, and when the stent is at the second diameter, the second locking member engages the first locking member such that the stent is prevented from moving from the second diameter to the first diameter.

All features described herein are independent of one another and, except where structurally impossible, can be used in combination with any other feature described herein. For example, the delivery apparatus 700 as shown in FIG. 9A can be used in combination with any of the prosthetic heart valves described herein, in particular, any of the valve frame configurations illustrated in FIGS. 1-8. In another example, the actuator configuration of FIG. 7A can be used in combination with any of the prosthetic heart valves described herein, in particular, any of the valve frame configurations illustrated in FIGS. 1-6C. In still another example, the actuator configuration of FIGS. 7B-7C can be used in combination with any of the prosthetic heart valves described herein, in particular, any of the valve frame configurations illustrated in FIGS. 1-6C. In yet another example, the actuator configuration of FIG. 8 can be used in combination with any of the prosthetic heart valves described herein, in particular, any of the valve frame configurations illustrated in FIGS. 1-6C. Indeed, any of the features illustrated or described with respect to FIGS. 1-10E and Examples 1-189 can be combined with any other feature illustrated or described with respect to FIGS. 1-10E and Examples 1-189 to provide systems, methods, devices, and implementations not otherwise illustrated or specifically described herein.

In view of the many possible implementations to which the principles of the disclosed technology may be applied, it should be recognized that the illustrated implementations are only preferred examples and should not be taken as limiting the scope of the disclosed technology. Rather, the scope of the invention is defined by the following claims. We therefore claim as our invention all that comes within the scope and spirit of these claims.

1. A prosthetic heart valve comprising:
a valvular structure comprising a plurality of leaflets; and
a frame configured to support the valvular structure and to move between a radially-compressed configuration and a radially-expanded configuration, the frame comprising a first axial end portion, a second axial end portion, a longitudinal axis extending from the first axial end portion to the second axial end portion, a support arm having a fixed end portion extending from the first axial end portion and a free end portion extending towards the second axial end portion, the free end portion rotationally offset relative to the fixed end portion, the free end portion comprising a first locking member, the frame having a second locking member that is spaced apart from the first locking member in a direction parallel to the longitudinal axis when the frame is in the radially-compressed configuration and that engages the first locking member when the frame is in the radially-expanded configuration.

2. The prosthetic heart valve of claim 1, wherein the frame comprises a plurality of struts defining a plurality of cells distributed circumferentially around the frame, and wherein the support arm, the first locking member, and the second locking member are integrated with the plurality of struts.

3. The prosthetic heart valve of claim 2, wherein the plurality of cells comprises a first cell and an inner cell disposed within the first cell, and wherein the first locking member and the second locking member are coupled to the inner cell.

4. The prosthetic heart valve of claim 1, wherein the free end portion is rotationally offset relative to the fixed end portion by 90 degrees or a multiple of 90 degrees.

5. The prosthetic heart valve of claim 1, wherein the first locking member comprises a first jaw and a second jaw spaced from each other by a gap, wherein the first jaw and the second jaw include one or more teeth protruding into the gap; and
wherein the second locking member includes one or more openings or recesses configured to receive the one or more teeth of the first locking member when the frame is in the radially-expanded configuration.

6. The prosthetic heart valve of claim 5, wherein the free end portion is rotationally offset relative to the fixed end portion such that the first jaw is disposed radially inwardly relative to the second jaw.

7. The prosthetic heart valve of claim 1, further comprising a rotatable actuation member coupled to the frame, wherein rotating the rotatable actuation member in a first direction relative to the frame increases a diameter of the frame, and wherein rotating the rotatable actuation member in a second direction relative to the frame decreases the diameter of the frame.

8. The prosthetic heart valve of claim 1, further comprising a lumen extending through the frame and configured to receive an actuation member of a delivery apparatus that can be releasably coupled to the first axial end portion the frame of the prosthetic heart valve, wherein moving the actuation member of the delivery apparatus in a first axial direction relative to the second axial end portion of the frame increases a diameter of the frame, and wherein moving the actuation member of the delivery apparatus in a second axial direction relative to the second axial end portion of the frame decreases the diameter of the frame.

9. The prosthetic heart valve of claim 1, wherein the frame is formed of a shape memory material, and wherein the frame is shape set to have a memorized configuration that is intermediate between the radially-compressed and radially-expanded configurations.

10. An assembly comprising:
a delivery apparatus comprising an elongated shaft; and
the prosthetic heart valve of claim 1 releasably supported within the delivery apparatus in the radially-compressed configuration for delivery into a patient's body.

11. A method of implanting a prosthetic heart valve in a patient's body, the method comprising:
inserting a distal end of a delivery apparatus into vasculature of a patient, the delivery apparatus comprising an elongated shaft, the prosthetic heart valve of any one of claims 1-9 being releasably supported within the delivery apparatus in the radially-compressed configuration;
advancing the prosthetic heart valve to a desired implantation site; and
using the delivery apparatus to expand the prosthetic heart valve to the radially-expanded configuration, thereby implanting the prosthetic heart valve at the desired implantation site.

12. The method of claim 11, further comprising prior to using the delivery apparatus to expand the prosthetic heart valve to the radially-expanded configuration, deploying the prosthetic heart valve from the delivery apparatus such that the prosthetic heart valve self-expands to a previously shape-set configuration that is intermediate between the radially-compressed and radially expanded configurations.

13. A method of fabricating a prosthetic heart valve, the method comprising:
forming a frame having a plurality of first cells and a plurality of first locking members, each first cell comprising a plurality of interconnected first struts and a second locking member at or adjacent to the second end of the respective first cell, each first locking member being at an end of a support arm that extends from a first end of a corresponding first cell, the frame being formed of a shape memory material, each first locking member being in an initial orientation; and
shape-setting each first locking member to have a twisted orientation with respect to the corresponding first cell.

14. The method of claim 13, wherein the shape-setting comprises:
at a temperature in excess of a transition temperature of the shape memory material, rotating each first locking member about a longitudinal axis of the support arm, such that the first locking member has the twisted orientation, with a first part of the first locking member disposed on one side of a plane of the respective first cell and a second part of the first locking member disposed on an opposite of the plane of the respective first cell; and
cooling the frame to a temperature below the transition temperature to set each first locking member in the twisted orientation.

15. The method of claim 14, wherein each first locking member is rotated by 90 degrees or a multiple of 90 degrees about the longitudinal axis of the corresponding support arm to provide the first locking member with the twisted orientation.

16. The method of claim 13, wherein the shape-setting comprises:
heating one or more portions of the frame to a temperature in excess of a transition temperature of the shape memory material;
at the temperature in excess of the transition temperature, rotating each first locking member about a longitudinal axis of the respective support arm by an incremental amount;
cooling the frame to a temperature below the transition temperature; and
repeating the heating, the rotating, and the cooling one or more times until each first locking member is provided with the twisted orientation.

17. The method of claim 13, wherein:
after the forming the frame, each first locking member is outside a boundary formed by the interconnected first struts of the corresponding first cell such that the support arm extends away from the second end of the first cell; and
the method further comprises, prior to or after the shape-setting each first locking member, rotating each support arm about the first end of the corresponding first cell such that at least part of the first locking member is within the boundary formed by the interconnected first struts and the support arm extends toward the second end of the corresponding first cell.

18. The method of claim 13, wherein the frame is formed as a flat structure with a linear array of first cells, and the method further comprises joining the first cells at opposite ends of the linear array to form an annular structure.

19. The method of claim 13, wherein the forming comprises cutting a solid starting material to form the struts of the first cells, the first locking members, the support arms, and the second locking members.

20. The method of claim 13, further comprising:
coupling a valvular structure to the frame, the valvular structure comprising a plurality of leaflets.
